(19) United States
(12) Patent Application Publication
(10) Pub. No.: US 2023/0104315 A1
(43) Pub. Date: Apr. 6, 2023

(54) METHOD OF IDENTIFYING EXTRACHROMOSOMAL DNA SIGNATURES

(71) Applicant: Boundless Bio, Inc., San Diego, CA (US)

(72) Inventors: Nam-Phuong NGUYEN, San Diego, CA (US); Jason CHRISTIANSEN, San Diego, CA (US); Laurence JADIN, San Diego, CA (US)

(21) Appl. No.: 17/907,147

(22) PCT Filed: Mar. 25, 2021
(86) PCT No.: PCT/US2021/024242

§ 371 Date: Sep. 23, 2022

Related U.S. Application Data
(60) Provisional application No. 63/023,731, filed on May 12, 2020.
(60) Provisional application No. 63/001,150, filed on Mar. 27, 2020.

(51) Int. Cl. C12Q 1/6886

(52) U.S. Cl. CPC C12Q 1/6886; C12Q 2600/106; C12Q 2600/112; C12Q 2600/156

(57) ABSTRACT

Provided herein are methods and systems for detecting ecDNA positive cancers and methods and systems for developing signatures of ecDNA positive cancers.

### CROSS-REFERENCE

This application claims the benefit of U.S. Provisional Patent Application No. 63/001,150, filed Mar. 27, 2020 and U.S. Provisional Patent Application No. 63/023,731, filed May 12, 2020, each of which is incorporated by reference herein in its entirety for all intents and purposes.

### BACKGROUND

Extrachromosomal DNA (ecDNA) is circular DNA that exists in the cell nucleus, separate from the chromosomes. It is a common mechanism for amplification of oncogenes, allowing cells in a tumor to reach high copy numbers of oncogenes. As a result, ecDNA drives heterogeneity in oncogene copy number amongst cells in a tumor. In some cases, the presence of ecDNA in a tumor has been associated with treatment outcomes and patient prognosis.

### SUMMARY

Provided herein are methods of treating a cancer. In some embodiments, the method comprises obtaining biomarker data for a biological sample derived from a subject diagnosed or suspected of having a cancer. In some embodiments, the method comprises classifying the ecDNA status of the sample as ecDNA positive or ecDNA negative. In some embodiments, the method comprises determining a treatment plan on the basis of the ecDNA status. In some embodiments, the treatment plan excludes the administration of an immune checkpoint inhibitor when the sample is ecDNA positive. In some embodiments, the treatment plan excludes the administration of an immune checkpoint inhibitor or excludes administration of an immune checkpoint inhibitor as a first line therapy when the sample is ecDNA positive. In some embodiments, the treatment plan includes the administration of an immune checkpoint inhibitor when the sample is ecDNA negative. In some embodiments, the biomarker data is selected from the group consisting of nucleic acid copy number, co-amplified genes, gene fusions, fragment length, SNP/SNV frequency, indel frequency, indel location, a functional pathway mutation, microsatellite instability, tumor inflammation score, PD-L1 expression, or tumor mutational burden.

Further provided herein are methods of classifying potential for responsiveness of a subject to immune checkpoint therapy. In some embodiments, the method comprises determining an ecDNA positive status of a sample taken from the subject, wherein the ecDNA positive status is determined based on a threshold of an ecDNA signature. In some embodiments, the method comprises classifying the subject as potentially responsive to immune checkpoint therapy when the sample is below threshold for the ecDNA positive status. Additionally provided herein are methods of classifying potential for non-responsiveness of a subject to immune checkpoint therapy, comprising determining the ecDNA positive status of a sample taken from the subject, wherein the ecDNA positive status is determined based on a threshold of an ecDNA signature; and classifying the subject as potentially nonresponsive to immune checkpoint therapy when the sample is above the threshold for the ecDNA positive status. In some embodiments, the ecDNA signature is selected from the group consisting of nucleic acid copy number, co-amplified genes, gene fusions, fragment length, SNP/SNV frequency, indel frequency, indel location, a functional pathway mutation, microsatellite instability, tumor inflammation score, PD-L1 expression, or tumor mutational burden. In some embodiments, the sample comprises tumor cells, circulating tumor cells, circulating vesicles, or circulating tumor DNA. In some embodiments, the sample is blood, serum, plasma, lymph, pleural effusion, saliva, urine, stool, tissue, resected tumor, or a combination thereof.

Further provided herein are methods of detecting an ecDNA positive cancer. In some embodiments, the method comprises: determining a first frequency of base substitution or indels within a defined region of DNA from the sample. In some embodiments, the method comprises generating a comparison of the first frequency to a control frequency. In some embodiments, the method comprises classifying an ecDNA status of the sample based on the comparison. In some embodiments, an increase in the first frequency as compared to the control frequency indicates an ecDNA positive sample. In some embodiments, classifying an ecDNA status further comprises assessing presence of one or more ecDNA signatures and classifying the ecDNA status based on the one or more ecDNA signatures and the comparison of frequencies. In some embodiments, the one or more ecDNA signatures are selected from the group consisting of nucleic acid copy number, co-amplified genes, gene fusions, fragment length, SNP/SNV frequency, indel frequency, indel location, a functional pathway mutation, microsatellite instability, tumor inflammation score, PD-L1 expression, or tumor mutational burden.

Additionally provided herein are methods of assessing progression of a cancer treatment. In some embodiments, the method comprises determining a first frequency of base substitutions or indels within a defined region of DNA from a first sample from a subject. In some embodiments, the method comprises determining a second frequency of base substitutions or indels within the defined region of DNA from a second sample from the subject, wherein the second sample is obtained after treatment with a targeted cancer therapy agent. In some embodiments, the method comprises comparing the first frequency to a second frequency to identify a change in the ecDNA status of the sample. In some embodiments, the change in ecDNA status comprises an increase in the second frequency as compared to the first frequency. In some embodiments, the method further comprises changing the cancer treatment based on the change in ecDNA status. In some embodiments, the sample comprises tumor cells, circulating tumor cells, circulating vesicles, or circulating tumor DNA. In some embodiments, the sample is blood, serum, plasma, lymph, pleural effusion, saliva, urine, stool, tissue, resected tumor, or a combination thereof.

Further provided herein are methods of assessing the potential for resistance to a cancer treatment. In some embodiments, the method comprises determining a first frequency and location of base substitution or indels within a defined region of DNA from a first sample from a subject. In some embodiments, the method comprises determining a second frequency and location of base substitution or indels within the defined region of DNA from a second sample from the subject, wherein the second sample is obtained during a course of treatment with a targeted cancer therapy agent. In some embodiments, the method comprises comparing the first frequency and location to a second frequency and location, wherein the subject is determined to have the potential for resistance to a cancer treatment when the second frequency or location is changed as compared to the first frequency or location. In some embodiments, the first sample, the second sample or both the first sample and second sample comprise tumor cells, circulating tumor cells, circulating vesicles, or circulating tumor DNA. In some embodiments, the first sample, the second sample, or both the first sample and the second sample are blood, serum, plasma, lymph, pleural effusion, saliva, urine, stool, tissue, resected tumor, or a combination thereof.

Provided herein are methods of detecting an ecDNA positive cancer in a subject. In some embodiments, the method comprises: (a) obtaining biomarker data for a biological sample derived from the subject; (b) processing the biomarker data from the subject with a database of control biomarkers; and (c) classifying the subject as having an ecDNA positive cancer when, based on the result of (b), the biomarker data from the subject is significantly different as compared to the database of control biomarkers. In some embodiments, the control biomarker data is derived from subjects that do not have cancer. In some embodiments, the control biomarker data is derived from subjects having ecDNA negative cancer. In some embodiments, the control biomarker data is a predetermined threshold value. In some embodiments, the biomarker data comprises one or more of nucleic acid copy number, co-amplified genes, gene fusions, fragment length, SNP/SNV frequency, indel frequency, indel location, microsatellite instability, tumor inflammation score, PD-L1 expression, or tumor mutational burden. In some embodiments, the biomarker data is measured by whole exome sequencing or targeted sequencing. In some embodiments, the tumor inflammation score is measured in a gene expression assay on a cancer cell from the subject. In some embodiments, the PD-L1 expression level is measured by a PD-L1 RNA gene expression assay on a cancer cell from the subject. In some embodiments, the PD-L1 expression level is measured by a PD-L1 protein expression assay on a cancer cell from the subject. In some embodiments, the microsatellite instability is measured by sequencing nucleic acids derived from a cancer cell from the subject. In some embodiments, the tumor mutational burden is measured by sequencing nucleic acids derived from a cancer cell from the subject. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer. In some embodiments, the method further comprises administering a therapeutic agent to the subject. In some embodiments, the therapeutic agent comprises atezolizumab, avelumab, cemiplimab, durvalumab, nivolumab, or pembrolizumab. In some embodiments, the therapeutic agent does not comprise an anti-PD-L1 or anti-PD-1 antibody.

Additionally provided herein are methods of detecting an ecDNA positive cancer in a subject, the method comprising: (a) obtaining a tumor inflammation score for a biological sample derived from the subject; (b) computer processing the tumor inflammation score from the subject with a control tumor inflammation score; and (c) classifying the subject as having an ecDNA positive cancer when, based on the result of (b), the tumor inflammation score from the subject is decreased as compared with the control tumor inflammation score. In some embodiments, the method further comprises: (d) obtaining a PD-L1 expression level for the biological sample derived from the subject; (e) computer processing the PD-L1 expression level from the subject with a control PD-L1 expression level; and (f) classifying the subject as having an ecDNA positive cancer when based on the results of (b) and (e), the tumor inflammation score from the subject is decreased as compared with the control tumor inflammation and the PD-L1 expression level is decreased as compared to the control PD-L1 expression level. In some embodiments, the control tumor inflammation score or the control PD-L1 expression level is derived from a subject that has ecDNA negative cancer. In some embodiments, the control tumor inflammation score or the control PD-L1 expression level is derived from a non-cancerous source. In some embodiments, the tumor inflammation score is measured in a gene expression assay on a cancer cell from the subject. In some embodiments, the PD-L1 expression level is measured by measuring a PD-L1 RNA level in a cancer cell from the subject. In some embodiments, the PD-L1 expression level is measured by measuring a PD-L1 protein level in a cancer cell from the subject. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer. In some embodiments, the cancer is gastric cancer. In some embodiments, the method further comprises administering a therapeutic agent to the subject. In some embodiments, the therapeutic agent comprises atezolizumab, avelumab, cemiplimab, durvalumab, nivolumab, or pembrolizumab. In some embodiments, the therapeutic agent does not comprise an anti-PD-L1 or anti-PD-1 antibody.

Further provided herein are methods of detecting an ecDNA positive cancer in a subject, the method comprising: (a) obtaining a PD-L1 expression level for the biological sample derived from the subject; (b) computer processing the PD-L1 expression level from the subject with a control PD-L1 expression level; and (c) classifying the subject as having an ecDNA positive cancer when based on the results of (b), the PD-L1 expression level is decreased as compared to the control PD-L1 expression level. In some embodiments, the method further comprises: (d) obtaining a tumor inflammation score for a biological sample derived from the subject; (e) computer processing the tumor inflammation score from the subject with a control tumor inflammation score; and (f) classifying the subject as having an ecDNA positive cancer when based on the result of (b) and (e), the PD-L1 expression level is decreased as compared to the control PD-L1 expression level and the tumor inflammation score from the subject is decreased as compared with the control tumor inflammation score. In some embodiments, the control tumor inflammation score or the control PD-L1 expression level is derived from a subject that has ecDNA negative cancer. In some embodiments, the control tumor inflammation score or the control PD-L1 expression level is derived from a non-cancerous source. In some embodiments, the PD-L1 expression level is measured by measuring a PD-L1 RNA level in a cancer cell from the subject. In some embodiments, the PD-L1 expression level is measured by measuring a PD-L1 protein level in a cancer cell from the subject. In some embodiments, the tumor inflammation score is measured in a gene expression assay on a cancer cell from the subject. In some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer. In some embodiments, the cancer is gastric cancer. In some embodiments, the method further comprises administering a therapeutic agent to the subject. In some embodiments, the therapeutic agent comprises atezolizumab, avelumab, cemiplimab, durvalumab, nivolumab, or pembrolizumab. In some embodiments, the therapeutic agent does not comprise an anti-PD-L1 or anti-PD-1 antibody.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention are utilized, and the accompanying drawings of which:

FIG. 1 shows an overview comparison of data for ecDNA detection between WGS and WES/targeted sequencing.

FIG. 2 shows the overlap between microsatellite instability (MSI) status and ecDNA positive cancers.

FIG. 3 shows the correlation of tumor inflammation score (TIS) with microsatellite instability (MSI), Epstein Barr Virus (EBV), genomic stability (GS), chromosomal instability (CIN), and ecDNA.

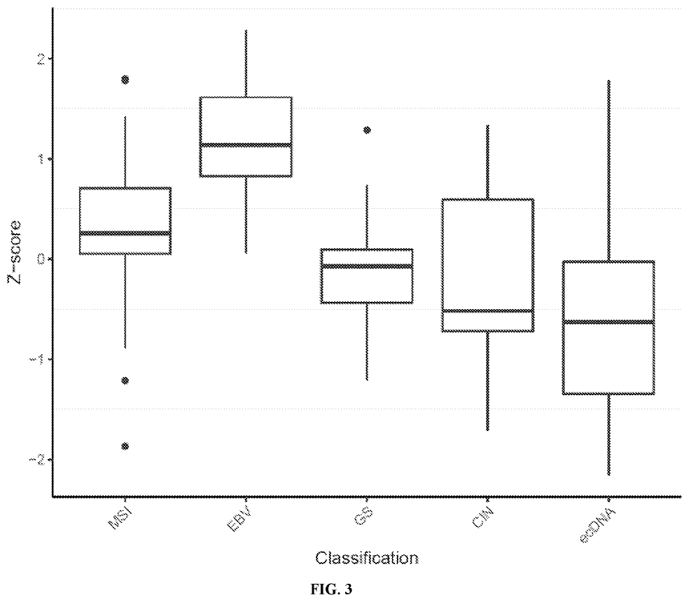

FIG. 4 shows the correlation of PD-L1 expression with microsatellite instability (MSI), Epstein Barr Virus (EBV), genomic stability (GS), chromosomal instability (CIN), and ecDNA.

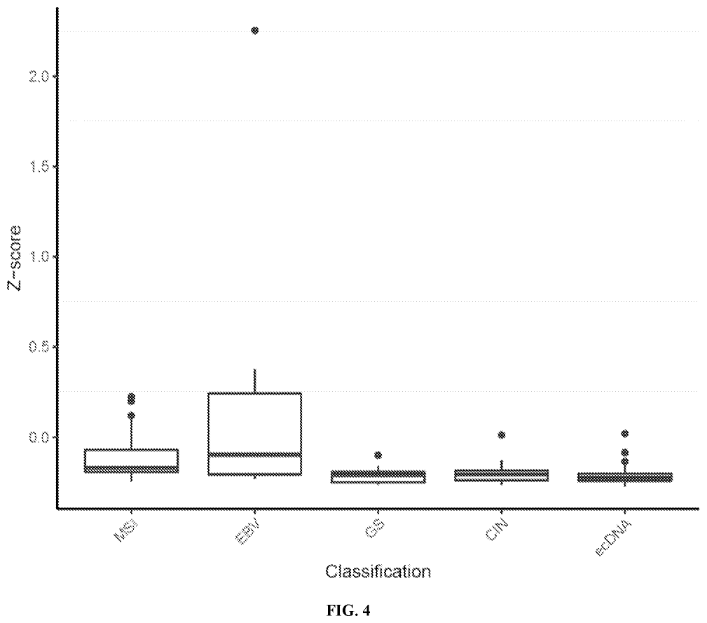

FIG. 5 shows the correlation of tumor mutational burden (TMB) with microsatellite instability (MSI), Epstein Barr Virus (EBV), genomic stability (GS), chromosomal instability (CIN), and ecDNA.

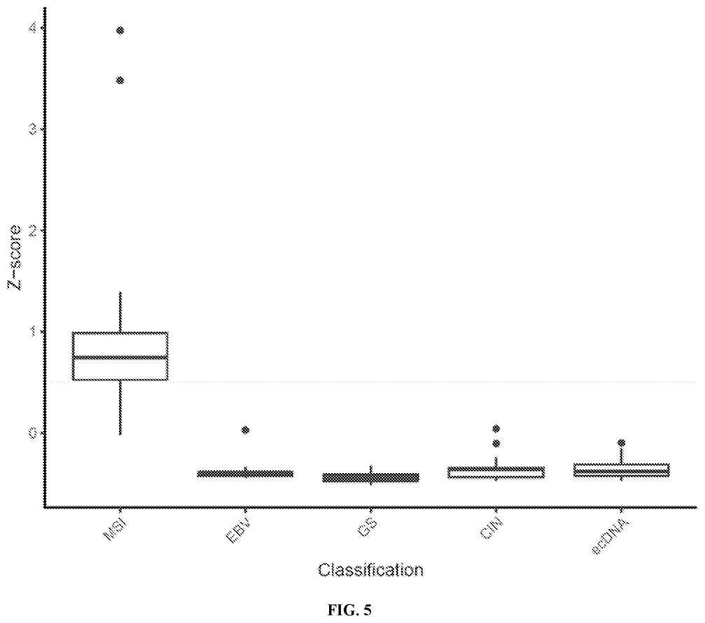

FIG. 6 shows a computer system that is programmed or otherwise configured to implement methods provided herein.

FIG. 7 shows ecDNA+vs. ecDNA− tumors stratified by MSI status. ** and **** indicate statistical significance as determined by chi-square test.

FIG. 8 shows the correlation of tumor inflammation score (TIS) with microsatellite instability (MSI), Epstein Barr Virus (EBV), genomic stability (GS), chromosomal instability (CIN), and ecDNA. *, ***, and **** indicate statistical significance as determined by Wilcoxon Rank Sum test.

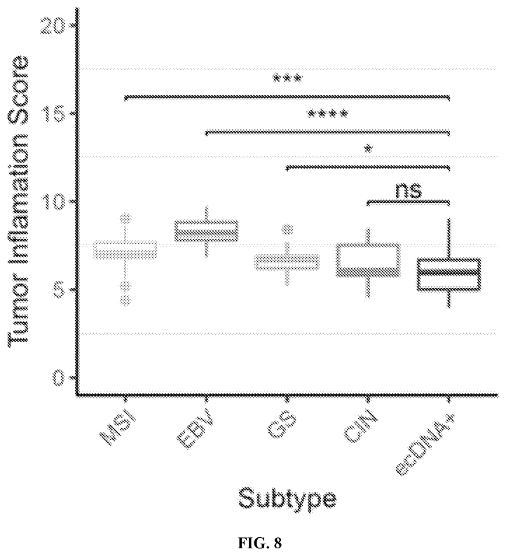

FIG. 9 shows the correlation of PD-L1 expression by mRNA with microsatellite instability (MSI), Epstein Barr Virus (EBV), genomic stability (GS), chromosomal instability (CIN), and ecDNA. ** and *** indicate statistical significance as determined by Wilcoxon Rank Sum test.

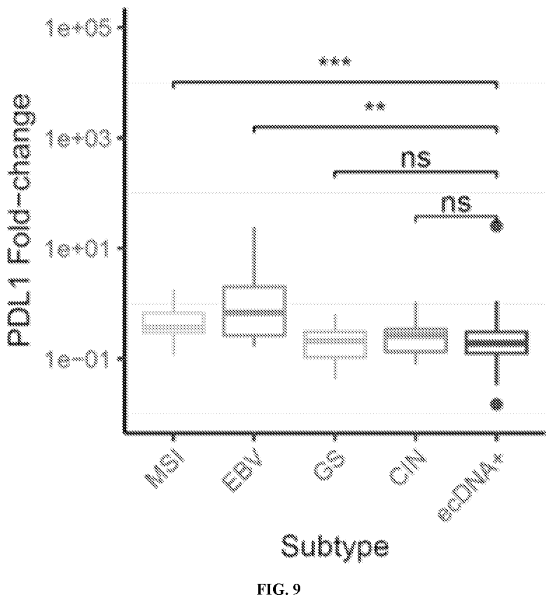

FIG. 10 shows the correlation of tumor mutational burden (TMB) with microsatellite instability (MSI), Epstein Barr Virus (EBV), genomic stability (GS), chromosomal instability (CIN), and ecDNA. **** indicates statistical significance as determined by Wilcoxon Rank Sum test.

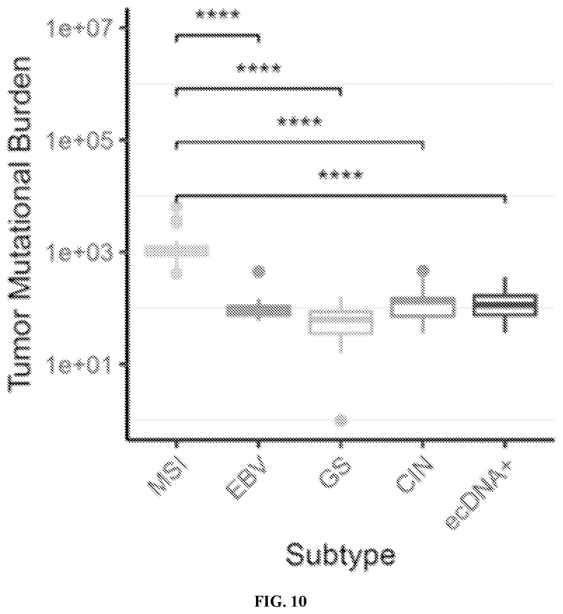

FIG. 11 shows ecDNA+ status across molecular subclasses.

FIG. 12 shows the number of patients from each subtype that have copy number variation (CNV) gain in oncogene hotspots. Patients in dark red have the CNV gain on the predicted ecDNA structure.

FIG. 13 shows proportion of ecDNA+ and ecDNA− enrichment for the selection of patients that are predicted to be responders or non-responders of pembrolizumab using combinations of biomarkers.

FIG. 14 shows a flow diagram for analysis of ecDNA signatures.

FIG. 15 shows, in the upper panel, the structure of an ecDNA-containing amplicon present in SNU16 cells as determined using AmpliconArchitect. In the lower panel of FIG. 15, average depth is shown by a line, insertion/deletion (indel) rate is shown by upward bars and substitution rate is shown by downward bars for three genomic regions corresponding to ecDNA as per AmpliconArchitect output.

FIG. 16 shows, in the upper panel, location of substitutions in three genomic regions corresponding to ecDNA in SNU16 cells, and their surrounding regions. Each vertical bar represents a unique substitution. Horizontal bars highlight the location of the ecDNA-containing amplicon. In the lower panel of FIG. 16, density of substitutions is shown in three genomic regions corresponding to ecDNA in SNU16 cells, and their surrounding regions (matching genomic coordinates shown in FIG. 16 upper panel).

FIG. 17 shows, in the upper panel, locations of indels in three genomic regions corresponding to ecDNA in SNU61 cells and their surrounding regions. Each vertical bar represents a unique insertion or deletion. The lower panel of FIG. 17 shows the density of indels in three genomic regions corresponding to ecDNA in SNU16 cells and their surrounding regions (matching genomic coordinates show in in FIG. 17 upper panel).

FIG. 18 shows substitution and indel rates calculated over the genomic region corresponding to the SNU16 ecDNA amplicon (open dot), as well as over 500 random equally sized continuous genomic regions (closed dots) plotted against average read depth (FIG. 18 left panel). The right panel of FIG. 18 shows average depth (line), indel rate (upward bars), and substitution rate (downward bars) per million nucleotides (1e5 sliding window).

FIG. 19 shows exome data obtained from cells before and after treatment with infigratinib or erlotinib.

FIG. 20 shows, in the upper panel, the structure of an ecDNA-containing amplicon present in H2170 squamous cell carcinoma cells (ATCC) as determined using AmpliconArchitect. In the lower panel of FIG. 20, average depth (line) indel rate (upward bars), and substitution rate (downward bars) are shown per million nucleotides (1e5 sliding window) in genomic regions corresponding to ecDNA as per AmpliconArchitect output.

### DETAILED DESCRIPTION

Extrachromosomal DNA (ecDNA) has been proposed as a mechanism for focal amplification of oncogenes, a key driver for tumorigenesis. Much of the current data identifying ecDNA in patients and model systems rely on the use of whole genome sequencing (WGS) to identify the presence/absence of ecDNA and its structure (e.g., whether it includes a driver oncogene). Such an approach allows software to examine the entire genomic context, including intragenic spaces, non-coding regions, etc. to identify complex ecDNA structures. However, sequencing in broader use, and particularly in the clinical oncology setting, is typically performed using, at most, whole exome sequencing (WES), and more commonly includes the use of only targeted panels, which focus on up to several hundred genes of interest. Because there is information lost when switching from WGS to WES or to targeted panels, current informatics pipelines are not sufficient to identify ecDNA, as such pipelines focus on genes and local mutations and not necessarily on the surrounding genomic context that WGS offers.

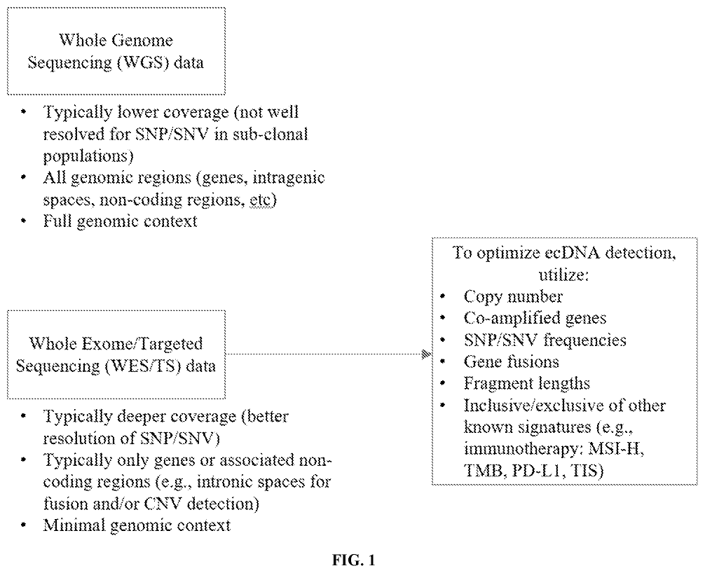

To address these limitations, provided herein is the development of signatures that can help improve the identification of ecDNA in non-WGS panels where the context is limited. These signatures take a broad approach to ecDNA identification by looking at a combination of gene copy number, gene fusions, fragment length, allele frequency, genomic location of amplification, and exclusivity with other potential biomarkers of response that are indicative of lack of ecDNA presence (FIG. 1).

Provided herein are methods and systems for detecting ecDNA positive cancer. Also provided herein are methods and systems for developing signatures of ecDNA positive cancer. These methods and systems, in some cases, use one or more biomarker data alone or in combination to determine whether a subject has ecDNA positive cancer.

### Methods of Detecting Extrachromosomal DNA Positive Cancer

In one aspect, provided herein are methods of detecting an extrachromosomal DNA (ecDNA) positive cancer in a subject. In some cases, the method comprises obtaining biomarker data for a biological sample derived from the subject. Next, the biomarker data from the subject is processed with a database of control biomarkers. In some embodiments, the subject then is classified as having an ecDNA positive cancer when the biomarker data from the subject is significantly different as compared to the database of control biomarkers. In some cases, the control biomarker data is derived from subjects having ecDNA negative cancer. In some cases, the control biomarker data is a predetermined threshold value. In some cases, the control biomarker data is derived from subjects not having cancer.

In another aspect, there are provided methods of detecting an ecDNA positive cancer comprising: determining a first frequency of base substitution or indels within a defined region of DNA from the sample; comparing the first frequency to a control frequency; and classifying an ecDNA status of the sample based on the comparison of frequencies. In some cases, an increase in the first frequency as compared to the control frequency indicates an ecDNA positive sample. In some cases, the classifying step further comprises assessing the presence of one or more ecDNA biomarker signatures and classifying the ecDNA status based on the one or more ecDNA signatures and the comparison of frequencies. In some cases, the one or more ecDNA signatures are selected from the group consisting of nucleic acid copy number, co-amplified genes, gene fusions, fragment length, SNP/SNV frequency, indel frequency, indel location, a functional pathway mutation, microsatellite instability, tumor inflammation score, PD-L1 expression, or tumor mutational burden. In some cases, a functional pathway mutation comprises a mutation in one or more functional pathway genes selected from the group consisting of TP53, MDM2, MDM4, CDKN2A (ARF), PTEN, AKT1, and TP53BP1.

In methods of detecting an ecDNA positive cancer provided herein, in some embodiments, the biomarker data comprises one or more of nucleic acid copy number, co-amplified genes, gene fusions, fragment length, SNP/SNV frequency, indel frequency, indel location, microsatellite instability, tumor inflammation score, PD-L1 expression, or tumor mutational burden. In some embodiments, the biomarker data comprises tumor inflammation score. In some embodiments, the biomarker data comprises PD-L1 expression. In some embodiments, the biomarker data comprises tumor inflammation score and PD-L1 expression.

In methods of detecting an ecDNA positive cancer provided herein, in some embodiments, the biomarker data is measured by whole exome sequencing or targeted sequencing. In some embodiments, the biomarker data is measured by whole genome sequencing. In some embodiments, the biomarker data is measured by a gene expression assay. In some embodiments, the biomarker data is measured by an RNA gene expression assay. In some embodiments, the biomarker data is measured by a protein expression assay. In some embodiments the biomarker data is measured by an accessible chromatin assay.

In methods of detecting an ecDNA positive cancer provided herein, in some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer. In some embodiments, the cancer is a solid tumor. In some embodiments, the cancer is a blood cancer. In some embodiments, the cancer is gastric cancer.

In methods of detecting an ecDNA positive cancer provided herein, in some embodiments, the method further comprises administering a therapeutic agent to the subject. In some embodiments, the method further comprises administering a checkpoint inhibitor. In some embodiments, the method further comprises administering a chemotherapeutic agent. In some embodiments, the method further comprises administering a PD-L1 inhibitor or a PD-1 inhibitor. In some embodiments, the method further comprises administering a PD-L1 inhibitor or a PD-1 inhibitor when an ecDNA positive cancer is not detected. In some embodiments, the method further comprises administering atezolizumab, avelumab, cemiplimab, durvalumab, nivolumab, or pembrolizumab. In some embodiments, the method further comprises administering a therapeutic agent that is not a PD-1 inhibitor or PD-L1 inhibitor. In some embodiments, the method further comprises administering a therapeutic agent that is not a PD-1 inhibitor or PD-L1 inhibitor when an ecDNA positive cancer is detected.

### Methods of Developing Signatures of Extrachromosomal DNA Positive Cancer

In another aspect, provided herein are methods of developing one or more signatures of an extrachromosomal DNA (ecDNA) positive cancer. In some cases, the method comprises obtaining biomarker data for a biological sample derived from a subject having an ecDNA positive cancer. Next, the biomarker data from the subject is processed with a database of control biomarkers. In some embodiments, the biomarker data is then classified as having an association with ecDNA positive cancer when the biomarker data from the subject is significantly different as compared to the database of control biomarkers. In some cases, the control biomarkers are derived from subjects having ecDNA negative cancer. In some cases, the control biomarkers are a predetermined threshold value. In some cases, the control biomarkers are derived from subjects not having cancer.

In methods of developing one or more signatures of an extrachromosomal DNA (ecDNA) positive cancer provided herein, in some embodiments, the biomarker data comprises one or more of nucleic acid copy number, co-amplified genes, functional pathway mutations, gene fusions, fragment length, SNP/SNV frequency, microsatellite instability, tumor inflammation score, PD-L1 expression, or tumor mutational burden. In some embodiments, the biomarker data comprises tumor inflammation score. In some embodiments, the biomarker data comprises PD-L1 expression. In some embodiments, the biomarker data comprises tumor inflammation score and PD-L1 expression.

In methods of developing one or more signatures of an extrachromosomal DNA (ecDNA) positive cancer provided herein, in some embodiments, the biomarker data is measured by whole exome sequencing or targeted sequencing. In some embodiments, the biomarker data is measured by whole genome sequencing. In some embodiments, the biomarker data is measured by a gene expression assay. In some embodiments, the biomarker data is measured by a RNA gene expression assay. In some embodiments, the biomarker data is measured by a protein expression assay. In some embodiments, the biomarker data is measured by an accessible chromatin assay.

In methods of developing one or more signatures of an extrachromosomal DNA (ecDNA) positive cancer provided herein, in some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer. In some embodiments, the cancer is a solid tumor. In some embodiments, the cancer is a blood cancer. In some embodiments, the cancer is gastric cancer.

### Methods of Treating Cancer

In some cases, detection of ecDNA in patient tumors informs new treatment paradigms to improve outcomes. Accordingly, in an aspect, provided herein are methods of treating a cancer. In some cases, the method comprises obtaining biomarker data for a biological sample derived from a subject diagnosed with a cancer. In some cases, the method comprises obtaining biomarker data for a biological sample derived from a subject suspected of having a cancer. In some cases, the method comprises classifying an ecDNA status of the sample as ecDNA positive or ecDNA negative. In some cases, the method comprises determining a treatment plan on the basis of the ecDNA status. In some cases, when the sample is ecDNA positive, the treatment plan excludes administration of an immune checkpoint inhibitor as a therapy or as a first-line therapy. In some cases, when the sample is ecDNA negative, the treatment plan includes administration of an immune checkpoint inhibitor.

In another aspect, there are provided methods of classifying a potential for responsiveness of a subject to immune checkpoint therapy. In some cases, the method comprises determining the ecDNA positive status of a biological sample taken from the subject. In some cases, the ecDNA positive status is determined based on a threshold of an ecDNA signature. In some cases, the method comprises classifying the subject as potentially responsive to immune checkpoint therapy when the ecDNA signature of the sample is below threshold for the ecDNA positive status.

In a further aspect, there are provided methods of classifying a potential for non-responsiveness of a subject to immune checkpoint therapy. In some cases, the method comprises determining an ecDNA positive status of a biological sample taken from the subject. In some cases, the ecDNA positive status is determined based on a threshold of an ecDNA signature. In some cases, the method comprises classifying the subject as potentially non-responsive to immune checkpoint therapy when the ecDNA signature of the sample is above threshold for the ecDNA positive status.

In embodiments, of methods of treatment provided herein, the sample comprises tumor cells, circulating tumor cells, circulating vesicles, circulating tumor DNA, or a combination thereof. In some embodiments, the sample is blood, serum, plasma, lymph, saliva, urine, stool, tissue, resected tumor, or a combination thereof.

In methods of treating cancer provided herein, in some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer. In some embodiments, the cancer is a solid tumor. In some embodiments, the cancer is a blood cancer. In some embodiments, the cancer is gastric cancer.

### Methods of Assessing Treatment

In an aspect, there are provided methods of assessing progression of a cancer treatment. In some cases, the method comprises determining a first frequency and/or location of base substitutions or indels within a defined region of DNA from a first sample from a subject. In some cases, the method comprises determining a second frequency and/or location of base substitutions or indels within the defined region of DNA from a second sample from the subject. In some cases, the second sample is obtained after treatment with a targeted cancer therapy agent. In some cases, the method comprises comparing the first frequency and/or location to the second frequency and/or location to identify a change in the ecDNA status of the sample. In some cases, the change in ecDNA status comprises an increase in second frequency as compared to the first frequency. In some cases, the change in ecDNA status comprises a change in the second frequency and location as compared to the first frequency and location. In some cases, the method further comprises changing the cancer treatment based on the change in ecDNA status.

In another aspect, there are provided methods of assessing potential for resistance to a cancer treatment. In some cases, the method comprises determining a first frequency of base substitutions or indels within a defined region of DNA from a first sample from a subject. In some cases, the method comprises determining a second frequency of base substitutions or indels within the defined region of DNA from a second sample from the subject. In some cases, the second sample is obtained during a course of treatment with a targeted cancer therapy agent. In some cases, the method comprises comparing the first frequency to a second frequency, wherein the second frequency is increased as compared to the first frequency. In some cases, the subject is determined to have potential for resistance to the cancer treatment based on the comparison.

In embodiments of methods of assessing treatment provided herein, the sample comprises tumor cells, circulating tumor cells, circulating vesicles, circulating tumor DNA, or a combination thereof. In some embodiments, the sample is blood, serum, plasma, lymph, saliva, urine, stool, tissue, resected tumor, or a combination thereof.

In methods of assessing treatment of cancer provided herein, in some embodiments, the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer. In some embodiments, the cancer is a solid tumor. In some embodiments, the cancer is a blood cancer. In some embodiments, the cancer is gastric cancer.

### Methods of Sequencing

According to some embodiments, polynucleotides (or amplification products thereof, which in some cases, have optionally been enriched) are subjected to a sequencing reaction to generate sequencing reads. In some cases, the sequencing is whole genome sequencing. In some cases, the sequencing is whole exome sequencing. In some cases, the sequencing is targeted sequencing. In some embodiments, sequencing reads produced by such methods are used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLiD®, etc.), Roche's 454 Life Sciences systems, Pacific Biosciences systems, Oxford Nanopore Technologies, nanoball sequencing, sequencing by hybridization, polymerized colony (POLONY) sequencing, nanogrid rolling circle sequencing (ROLONY), etc. In some embodiments, sequencing comprises the use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product.

In some embodiments, sequencing methods of the present disclosure provide information useful for various applications, such as, for example, identifying a disease (e.g., cancer) in a subject or determining that the subject has ecDNA positive cancer. In some cases, sequencing provides biomarker data for a cancer derived from a subject. In some cases, sequencing provides a determination of one or more of nucleic acid copy number, co-amplified genes, functional pathway mutations, gene fusions, fragment length, SNP/SNV frequency, indel frequency or location, microsatellite instability, tumor inflammation score, or tumor mutational burden for a cancer from a subject. In some cases, sequencing provides a sequence of a polymorphic region. In some cases, sequencing provides a determination of microsatellite instability in a cancer derived from a subject. In some cases, sequencing provides a determination of tumor mutational burden in a cancer derived from a subject. In some cases, sequencing provides a determination of tumor inflammation score in a cancer derived from a subject.

### Methods of Measuring Gene Expression and DNA Copy Number

According to embodiments herein, gene expression is contemplated to be measured using any suitable method. In some embodiments, gene expression is measured by quantifying the amount of RNA. In some embodiments, gene expression is measured by quantifying the amount of protein. In some embodiments, gene expression is measured by quantifying the number of cells expressing an RNA or a protein by suitable methods.

In some embodiments of methods herein, gene expression is measured by quantifying the amount of RNA for one or more genes. In some embodiments, RNA expression is measured by one or more methods, including, but not limited to, RT-PCR, quantitative RT-PCR, real time RT-PCR, Northern blot, RNA sequencing, in situ hybridization, and other suitable methods. In some embodiments, a sample is homogenized and RNA is purified prior to quantifying gene expression. In some embodiments, purified RNA is subjected to reverse transcriptase to create cDNA prior to quantifying gene expression. In some embodiments, histologic sections are made from a sample, such as a tumor sample, and subjected to in situ hybridization using colorimetric, fluorescent, chemiluminescent, radioactive, or biotinylated probes to quantify gene expression. In some embodiments, cells of a sample, such as a tumor, are dissociated and fixed, prior to staining with a fluorescent nucleic acid probe to quantify the gene expression.

In some embodiments of methods herein, gene expression is measured by quantifying the amount of protein for one or more genes. In some embodiments, protein expression is measured by one or more methods, including, but not limited to, Western blot, FACS, immunohistochemistry, immunofluorescence, ELISA, ELISPOT, mass spectroscopy, tandem mass tag proteomics, and other suitable methods. In some embodiments, a sample is homogenized prior to quantifying gene expression. In some embodiments, protein is purified from the sample prior to quantifying gene expression. In some embodiments, a sample is subjected to SDS-PAGE and optionally transferred to a membrane prior to quantifying gene expression. In some embodiments, an antibody is used to quantify gene expression. In some embodiments, a sample is applied to an antibody-coated surface in order to quantify gene expression. In some embodiments, histologic sections are made from a sample, such as a tumor sample, an antibody is hybridized to the fixed sample, and the antibody binding is detected using fluorescent, chemiluminescent, radioactive, or biotinylated probes to quantify gene expression. In some embodiments, cells of a sample, such as a tumor, are dissociated and fixed, prior to staining with a fluorescent antibody probe to quantify the gene expression.

According to embodiments herein, copy number variation is contemplated to be measured using any suitable method. In some embodiments of methods herein, copy number variation is measured by quantifying the amount of DNA for one or more genes, or one or more loci. In some embodiments, copy number variation is measured by one or more methods, including, but not limited to, PCR, quantitative PCR, real time PCR, Southern blot, DNA sequencing, in situ hybridization, and other suitable methods. In some embodiments, a sample is homogenized, and DNA is purified prior to quantifying copy number variation. In some embodiments, histologic sections are made from a sample, such as a tumor sample, and subjected to in situ hybridization using colorimetric, fluorescent, chemiluminescent, radioactive, or biotinylated probes to quantify copy number variation. In some embodiments, cells of a sample, such as a tumor, are dissociated and fixed prior to staining with a fluorescent nucleic acid probe to quantify copy number variation.

### Samples

In some embodiments of the various methods described herein, the sample is from a subject. In some cases, a subject is any animal, including but not limited to, a cow, a pig, a mouse, a rat, a chicken, a cat, a dog, etc., and is usually a mammal, such as a human. Sample polynucleotides and/or proteins are often isolated from a tumor sample, a tissue sample, organ sample, or bodily fluid sample, including, for example, blood sample, or fluid sample containing nucleic acids (e.g., saliva). Other examples of sample sources include those from blood, urine, feces, nares, the lungs, the gut, other bodily fluids or excretions, materials derived therefrom, or combinations thereof. In some embodiments, the sample is a blood sample or a portion thereof (e.g., blood plasma or serum). In some embodiments, a sample from a single individual is divided into multiple separate samples (e.g., 2, 3, 4, 5, 6, 7, 8, 9, 10, or more separate samples) that are subjected to methods of the disclosure independently, such as analysis in duplicate, triplicate, quadruplicate, or more. Where a sample is from a subject, in some cases, the reference sequence is also derived from the subject, such as a consensus sequence from the sample under analysis or the sequence of polynucleotides from another sample or tissue of the same subject. For example, in some cases, a blood sample is analyzed for mutations, while cellular DNA from another sample (e.g., buccal or skin sample) is analyzed to determine the reference sequence.

Polynucleotides can be extracted from a sample according to any suitable method. A variety of kits are available for extraction of polynucleotides, selection of which, in some cases, depends on the type of sample, or the type of nucleic acid to be isolated. Examples of extraction methods are provided herein, such as those described with respect to any of the various aspects disclosed herein. In one example, the sample is a blood sample, such as a sample collected in an EDTA tube (e.g., BD Vacutainer). Plasma can be separated from the peripheral blood cells by centrifugation (e.g., 10 minutes at 1900×g at 4° C.). Plasma separation performed in this way on a 6 mL blood sample will typically yield 2.5 to 3 mL of plasma. Circulating cell-free DNA can be extracted from a plasma sample, such as by using a QIAmp Circulating Nucleic Acid Kit (Qiagen), according to the manufacturer's protocol. In some instances, DNA is then quantified (e.g., on an Agilent 2100 Bioanalyzer with High Sensitivity DNA kit (Agilent)). As an example, the yield of circulating DNA from such a plasma sample from a healthy person can range from 1 ng to 10 ng per mL of plasma, with significantly more in disease (e.g., cancer) patient samples.

### Cancer

Methods and systems provided herein, in certain aspects, allow for detecting extrachromosomal DNA (ecDNA) positive cancer and allow for developing signatures of ecDNA positive cancer. Examples of cancers that are contemplated in accordance with a method disclosed herein include, without limitation, acanthoma, acinic cell carcinoma, acoustic neuroma, acral lentiginous melanoma, acrospiroma, acute eosinophilic leukemia, acute lymphoblastic leukemia, acute megakaryoblastic leukemia, acute monocytic leukemia, acute myeloblastic leukemia with maturation, acute myeloid dendritic cell leukemia, acute myeloid leukemia, acute promyelocytic leukemia, adamantinoma, adenocarcinoma, adenoid cystic carcinoma, adenoma, adenomatoid odontogenic tumor, adrenocortical carcinoma, adult T-cell leukemia, aggressive NK-cell leukemia, AIDS-related cancers, AIDS-related lymphoma, alveolar soft part sarcoma, ameloblastic fibroma, anal cancer, anaplastic large cell lymphoma, anaplastic thyroid cancer, angioimmunoblastic T-cell lymphoma, angiomyolipoma, angiosarcoma, appendix cancer, astrocytoma, atypical teratoid rhabdoid tumor, basal cell carcinoma, basal-like carcinoma, B-cell leukemia, B-cell lymphoma, Bellini duct carcinoma, biliary tract cancer, bladder cancer, blastoma, bone cancer, bone tumor, brain stem glioma, brain lower grade glioma, brain tumor, breast cancer, breast invasive carcinoma, Brenner tumor, bronchial tumor, bronchioloalveolar carcinoma, brown tumor, Burkitt's lymphoma, cancer of unknown primary site, carcinoid tumor, carcinoma, carcinoma in situ, carcinoma of the penis, carcinoma of unknown primary site, carcinosarcoma, Castleman's disease, central nervous system embryonal tumor, cerebellar astrocytoma, cerebral astrocytoma, cervical cancer, cervical squamous cell carcinoma, cholangiocarcinoma, chondroma, chondrosarcoma, chordoma, choriocarcinoma, choroid plexus papilloma, chronic lymphocytic leukemia, chronic monocytic leukemia, chronic myelogenous leukemia, chronic myeloproliferative disorder, chronic neutrophilic leukemia, clear-cell tumor, colon adenocarcinoma, colon cancer, colorectal cancer, craniopharyngioma, cutaneous T-cell lymphoma, Degos disease, dermatofibrosarcoma protuberans, dermoid cyst, desmoplastic small round cell tumor, diffuse large B cell lymphoma, dysembryoplastic neuroepithelial tumor, embryonal carcinoma, endodermal sinus tumor, endometrial cancer, endometrial uterine cancer, endometrioid tumor, enteropathy-associated T-cell lymphoma, ependymoblastoma, ependymoma, epithelioid sarcoma, erythroleukemia, esophageal cancer, esophageal carcinoma, esthesioneuroblastoma, Ewing family of tumor, Ewing family sarcoma, Ewing's sarcoma, extracranial germ cell tumor, extragonadal germ cell tumor, extrahepatic bile duct cancer, extramammary fallopian tube cancer, fetus in fetu, fibroma, fibrosarcoma, follicular lymphoma, follicular thyroid cancer, gallbladder cancer, gallbladder cancer, ganglioglioma, ganglioneuroma, gastric cancer, gastric lymphoma, gastrointestinal cancer, gastrointestinal carcinoid tumor, gastrointestinal stromal tumor, gastrointestinal stromal tumor, germ cell tumor, germinoma, gestational choriocarcinoma, gestational trophoblastic tumor, giant cell tumor of bone, glioblastoma multiforme, glioma, gliomatosis cerebri, glomus tumor, glucagonoma, gonadoblastoma, granulosa cell tumor, hairy cell leukemia, head and neck cancer, heart cancer, hemangioblastoma, hemangiopericytoma, hemangiosarcoma, hematological malignancy, hepatocellular carcinoma, hepatosplenic T-cell lymphoma, hereditary breast-ovarian cancer syndrome, Hodgkin's lymphoma, hypopharyngeal cancer, hypothalamic glioma, inflammatory breast cancer, intraocular melanoma, islet cell carcinoma, islet cell tumor, juvenile myelomonocytic leukemia, Kaposi's sarcoma, kidney cancer, Klatskin tumor, Krukenberg tumor, laryngeal cancer, laryngeal cancer, lentigo maligna melanoma, leukemia, lip and oral cavity cancer, liposarcoma, liver hepatocellular carcinoma, lung adenocarcinoma, lung cancer, lung squamous cell carcinoma, luteoma, lymphangioma, lymphangiosarcoma, lymphoepithelioma, lymphoid leukemia, lymphoma, macroglobulinemia, lymphoid neoplasm diffuse large b-cell lymphoma, malignant fibrous histiocytoma, malignant fibrous histiocytoma of bone, malignant glioma, malignant mesothelioma, malignant peripheral nerve sheath tumor, malignant rhabdoid tumor, malignant triton tumor, MALT lymphoma, mantle cell lymphoma, mast cell leukemia, mediastinal germ cell tumor, mediastinal tumor, medullary thyroid cancer, medulloblastoma, medulloepithelioma, melanoma, meningioma, merkel cell carcinoma, mesothelioma, metastatic squamous neck cancer with occult primary, metastatic urothelial carcinoma, mixed Mullerian tumor, monocytic leukemia, mouth cancer, mucinous tumor, multiple endocrine neoplasia syndrome, multiple myeloma, mycosis fungoides, mycosis fungoides, myelodysplastic disease, myelodysplastic syndromes, myeloid leukemia, myeloid sarcoma, myeloproliferative disease, myxoma, nasal cavity cancer, nasopharyngeal cancer, nasopharyngeal carcinoma, neoplasm, neurinoma, neuroblastoma, neurofibroma, neuroma, nodular melanoma, non-Hodgkin lymphoma, nonmelanoma skin cancer, non-small cell lung cancer, ocular oncology, oligoastrocytoma, oligodendroglioma, oncocytoma, optic nerve sheath meningioma, oral cancer, oropharyngeal cancer, osteosarcoma, ovarian cancer, ovarian epithelial cancer, ovarian germ cell tumor, ovarian low malignant potential tumor, Paget's disease of the breast, pancoast tumor, pancreatic cancer, papillary thyroid cancer, papillomatosis, paraganglioma, paranasal sinus cancer, parathyroid cancer, penile cancer, perivascular epithelioid cell tumor, pharyngeal cancer, pheochromocytoma, pineal parenchymal tumor of intermediate differentiation, pineoblastoma, pituicytoma, pituitary adenoma, pituitary tumor, plasma cell neoplasm, pleuropulmonary blastoma, polyembryoma, precursor T-lymphoblastic lymphoma, primary central nervous system lymphoma, primary effusion lymphoma, primary hepatocellular cancer, primary liver cancer, primary peritoneal cancer, primitive neuroectodermal tumor, prostate cancer, pseudomyxoma peritonei, rectal cancer, renal cell carcinoma, respiratory tract carcinoma involving the NUT Gene on chromosome 15, retinoblastoma, rhabdomyoma, rhabdomyosarcoma, Richter's transformation, sacrococcygeal teratoma, salivary gland cancer, sarcoma, schwannomatosis, sebaceous gland carcinoma, secondary neoplasm, seminoma, serous tumor, Sertoli-Leydig cell tumor, sex cord-stromal tumor, Sezary syndrome, signet ring cell carcinoma, skin cancer, skin cutaneous melanoma, small blue round cell tumor, small cell carcinoma, small cell lung cancer, small cell lymphoma, small intestine cancer, soft tissue sarcoma, somatostatinoma, soot wart, spinal cord tumor, spinal tumor, splenic marginal zone lymphoma, squamous cell carcinoma, stomach adenocarcinoma, stomach cancer, superficial spreading melanoma, supratentorial primitive neuroectodermal tumor, surface epithelial-stromal tumor, synovial sarcoma, T-cell acute lymphoblastic leukemia, T-cell large granular lymphocyte leukemia, T-cell leukemia, T-cell lymphoma, T-cell prolymphocytic leukemia, teratoma, terminal lymphatic cancer, testicular cancer, thecoma, throat cancer, thymic carcinoma, thymoma, thyroid cancer, transitional cell cancer of renal pelvis and ureter, transitional cell carcinoma, urachal cancer, urethral cancer, urogenital neoplasm, urothelial bladder carcinoma, uterine corpus endometrial carcinoma, uterine sarcoma, uveal melanoma, vaginal cancer, Verner Morrison syndrome, verrucous carcinoma, visual pathway glioma, vulvar cancer, Waldenstrom's macroglobulinemia, Warthin's tumor, Wilms' tumor, and combinations thereof. In some cases, the cancer comprises glioblastoma multiforme, esophageal carcinoma, sarcoma, urothelial bladder carcinoma, lung squamous cell carcinoma, ovarian cancer, head and neck cancer, cervical squamous cell carcinoma, breast invasive carcinoma, lymphoid neoplasm diffuse large B-cell lymphoma, stomach adenocarcinoma, lung adenocarcinoma, skin cutaneous melanoma, uterine corpus endometrial carcinoma, liver hepatocellular carcinoma, brain lower grade glioma, or colon adenocarcinoma. In some cases, the cancer is responsive to checkpoint inhibitor therapy. In some cases, the cancer is not responsive to checkpoint inhibitor therapy. In some cases, the cancer is responsive to anti-PD-1 or anti-PD-L1 therapy. In some cases, the cancer is not responsive to anti-PD-1 or anti-PD-L1 therapy. In some cases, the cancer is a solid tumor. In some cases, the cancer is a blood cancer.

### Systems and Computer Assisted Methods

In additional aspects, there are provided systems for detecting an extrachromosomal DNA (ecDNA) positive cancer in a subject. In some embodiments, the system comprises one or more computer processors that are individually or collectively programmed to process biomarker data for a biological sample derived from the subject against a database of control biomarker data to classify the subject as having an ecDNA positive cancer when the biomarker data from the subject is significantly different as compared to the database of control biomarker data. In some cases, the control biomarker data is derived from subjects having ecDNA negative cancer. In some cases, the control biomarker data is a predetermined threshold value. In some cases, the control biomarker data is derived from subjects not having cancer.

In further aspects, there are provided methods of developing one or more signatures of an extrachromosomal DNA (ecDNA) positive cancer. In some embodiments, the system comprises one or more computer processors that are individually or collectively programmed to process a biomarker data for a biological sample derived from a subject having an ecDNA positive cancer against a database of control biomarker data to classify the biomarker data as having an association with ecDNA positive cancer when the biomarker data from the subject is significantly different as compared to the database of control biomarkers. In some cases, the control biomarker data is derived from subjects having ecDNA negative cancer. In some cases, the control biomarker data is a predetermined threshold value. In some cases, the control biomarker data is derived from subjects not having cancer.

Further provided herein are non-transitory computer-readable mediums. In some embodiments, a non-transitory computer-readable medium comprises machine-executable code that, upon execution by one or more computer processors, implements a method for detecting an extrachromosomal DNA (ecDNA) positive cancer in a subject comprising accessing a first database comprising biomarker data for a biological sample derived from a subject and accessing a second database comprising one or more control biomarkers. In some embodiments, the method comprises processing the biomarker data for the biological sample from the subject from the first database against the one or more control biomarker data from the second database to classify the biomarker data as having an association with ecDNA positive cancer when the biomarker data from the subject is significantly different as compared to the database of control biomarkers. In some cases, the control biomarker data is derived from subjects having ecDNA negative cancer. In some cases, the control biomarker data is a predetermined threshold value. In some cases, the control biomarker data is derived from subjects not having cancer.

In some embodiments, a non-transitory computer-readable medium comprises machine-executable code that, upon execution by one or more computer processors, implements a method of developing one or more signatures of an extrachromosomal DNA (ecDNA) positive cancer comprising accessing a first database comprising biomarker data for a biological sample derived from a subject having an ecDNA positive cancer and accessing a second database comprising one or more control biomarkers. In some embodiments, the method comprises processing the biomarker data for the biological sample from the subject from the first database against the one or more control biomarker data from the second database to classify the subject as having an ecDNA positive cancer when the biomarker data from the subject is significantly different as compared to the database of control biomarkers. In some cases, the control biomarker data is derived from subjects having ecDNA negative cancer. In some cases, the control biomarker data is a predetermined threshold value. In some cases, the control biomarker data is derived from subjects not having cancer.

A computer for use in the system can comprise one or more processors. In some embodiments, processors are associated with one or more controllers, calculation units, and/or other units of a computer system, or implanted in firmware as desired. If implemented in software, in some embodiments, the routines are stored in any computer-readable memory such as in RAM, ROM, flash memory, a magnetic disk, a laser disk, or other suitable storage media. Likewise, in some embodiments, this software is delivered to a computing device via any known delivery method including, for example, over a communication channel such as a telephone line, the internet, a wireless connection, etc., or via a transportable medium, such as a computer readable disk, flash drive, etc. In some embodiments, the various steps are implemented as various blocks, operations, tools, modules and techniques which, in turn, in some cases, are implemented in hardware, firmware, software, or any combination of hardware, firmware, and/or software. When implemented in hardware, in some cases, some or all of the blocks, operations, techniques, etc. are implemented in, for example, a custom integrated circuit (IC), an application-specific integrated circuit (ASIC), a field programmable logic array (FPGA), a programmable logic array (PLA), etc. A client-server, relational database architecture can be used in embodiments of the system. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments, the server computer handles all of the database functionality. The client computer can have software that handles all the front-end data management and can also receive data input from users.

The system can be configured to receive a user request to perform a detection reaction on a sample. In some embodiments, the user request is direct or indirect. Examples of direct requests include those transmitted by way of an input device, such as a keyboard, mouse, or touch screen. Examples of indirect requests include transmission via a communication medium, such as over the internet (either wired or wireless).

The system can further comprise an amplification system that performs a nucleic acid amplification reaction on the sample or a portion thereof in response to the user request. A variety of methods of amplifying polynucleotides (e.g., DNA and/or RNA) are available. In some embodiments, amplification is linear, exponential, or involves both linear and exponential phases in a multi-phase amplification process. In some cases, amplification methods involve changes in temperature, such as a heat denaturation step, or are isothermal processes that do not require heat denaturation. Non-limiting examples of suitable amplification processes are described herein, such as with regard to any of the various aspects of the disclosure. A variety of systems for amplifying polynucleotides are available, and, in some cases, vary based on the type of amplification reaction to be performed. For example, for amplification methods that comprise cycles of temperature changes, in some cases, the amplification system comprises a thermocycler. An amplification system can comprise a real-time amplification and detection instrument, such as systems manufactured by Applied Biosystems, Roche, and Strategene. Samples, polynucleotides, primers, polymerases, and other reagents can be any of those described herein, such as with regard to any of the various aspects. Systems can be selected and or designed to execute any such methods.

In some embodiments, systems further comprise a sequencing system that generates sequencing reads for polynucleotides amplified by the amplification system and identifies sequence differences between sequencing reads and a reference sequence. The sequencing system and the amplification system, in some embodiments, is the same, or comprise overlapping equipment. For example, both the amplification system and the sequencing system, in some cases, utilize the same thermocycler. A variety of sequencing platforms for use in the system are available, and, in some embodiments, are selected based on the selected sequencing method. Examples of sequencing methods are described herein. In some cases, amplification and sequencing involve the use of liquid handlers. Several commercially available liquid handling systems can be utilized to run the automation of these processes (see for example liquid handlers from Perkin-Elmer, Beckman Coulter, Caliper Life Sciences, Tecan, Eppendorf, Apricot Design, Velocity 11 as examples). A variety of automated sequencing machines are commercially available and include sequencers manufactured by Life Technologies (SOLiD platform, and pH-based detection), Roche (454 platform), Illumina (e.g., flow cell based systems, such as Genome Analyzer devices). In some cases, transfer between 2, 3, 4, 5, or more automated devices (e.g., between one or more of a liquid handler and a sequencing device) is manual or automated.

The system can further comprise a report generator that sends a report to a recipient, wherein the report contains results for detecting an extrachromosomal DNA (ecDNA) positive cancer in a subject. In some cases, a report is generated in real-time, such as during a sequencing read or while sequencing data is being analyzed, with periodic updates as the process progresses. In addition, or alternatively, a report is generated at the conclusion of the analysis. In some embodiments, the report is generated automatically, such as when the system completes the step of classifying the subject as having an ecDNA positive cancer. In some embodiments, the report is generated in response to instructions from a user. In addition to the results of classifying the subject as having an ecDNA positive cancer, in some cases, a report also contains an analysis based on the one or more biomarkers. For example, a suggestion based on this information (e.g., additional tests, monitoring, or remedial measures). The report can take any of a variety of forms. It is envisioned that data relating to the present disclosure can be transmitted over such networks or connections (or any other suitable approach for transmitting information, including but not limited to mailing a physical report, such as a print-out) for reception and/or for review by a receiver. The receiver can be but is not limited to an individual, or an electronic system (e.g., one or more computers, and/or one or more servers).

In alternate cases, the machine-readable medium comprising computer-executable code takes many forms, including but not limited to, a tangible storage medium, a carrier wave medium or physical transmission medium. Non-volatile storage media include, for example, optical or magnetic disks, such as any of the storage devices in any computers) or the like, such as to be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media, in some cases, takes the form of electric or electromagnetic signals, or acoustic or light waves such as those generated during radio frequency (RF) and infrared (IR) data communications. Common forms of computer-readable media, therefore, include for example a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer, in some cases, reads programming code and/or data. In some cases, many of these forms of computer-readable media are involved in carrying one or more sequences of one or more instructions to a processor for execution.

The subject computer-executable code can be executed on any suitable device comprising a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI or in the form of preprogrammed instructions, e.g., preprogrammed for a variety of different specific operations.

### Computer Systems

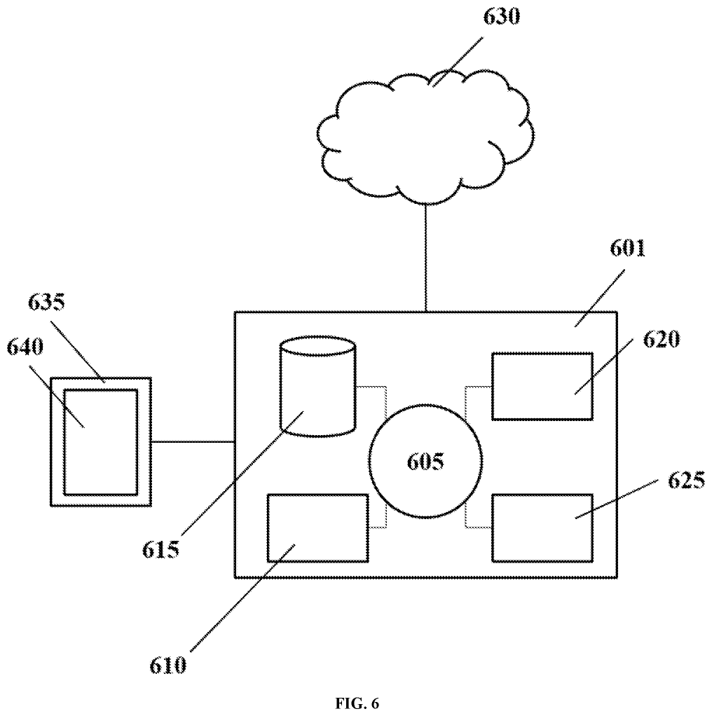

The present disclosure provides computer systems that are programmed to implement methods of the disclosure. FIG. 6 shows a computer system 601 that is programmed or otherwise configured to detect extrachromosomal DNA (ecDNA) positive cancer or develop signatures of ecDNA positive cancer. The computer system 601 can regulate various aspects of detection and analysis of biomarkers associated with ecDNA positive cancer disclosed in the present disclosure, such as, for example, obtaining biomarker data for a biological sample derived from the subject, processing the biomarker data from the subject with a database of control biomarker data or preset thresholds, and classifying the subject as having an ecDNA positive cancer when the biomarker data from the subject is significantly different as compared to the database of control biomarker data or preset thresholds. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi-core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which can enable devices coupled to the computer system 601 to behave as a client or a server.

The CPU 605 can execute a sequence of machine-readable instructions, which can be embodied in a program or software. In some cases, the instructions are stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., a healthcare provider or a patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods, as described herein can be implemented by way of machine (e.g., computer processor) executable code stored on an electronic storage location of the computer system 601, such as, for example, on the memory 610 or electronic storage unit 615. The machine-executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology can be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which, in some cases, provide non-transitory storage at any time for the software programming. All or portions of the software can at times be communicated through the Internet or various other telecommunication networks. Such communications, for example, in some cases, enable loading of the software from one computer or processor into another, from a management server or host computer into the computer platform of an application server. Thus, another type of media that, in some embodiments, bears the software elements includes optical, electrical and electromagnetic waves, such as used across physical interfaces between local devices, through wired and optical landline networks and over various air-links. The physical elements that carry such waves, such as wired or wireless links, optical links or the like, also, in some cases, are considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, in some cases, are used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media, in some embodiments, takes the form of electric or electromagnetic signals, or acoustic or light waves such as those generated during radio frequency (RF) and infrared (IR) data communications. Common forms of computer-readable media, therefore, include for example a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media, in some embodiments, are involved in carrying one or more sequences of one or more instructions to a processor for execution.

The computer system 601 can include or be in communication with an electronic display 635 that comprises a user interface (UI) 640 for providing, for example, results of methods of the present disclosure. Examples of UIs include, without limitation, a graphical user interface (GUI) and a web-based user interface.

Methods and systems of the present disclosure can be implemented by way of one or more algorithms. An algorithm can be implemented by way of software upon execution by the central processing unit 605. The algorithm can be, for example, a trained algorithm (or trained machine learning algorithm), such as, for example, a support vector machine or neural network.

### Definitions

As used herein the term “about” or “approximately” means within an acceptable error range for the particular value as determined by one of ordinary skill in the art, which can depend in part on how the value is measured or determined, i.e., the limitations of the measurement system. For example, “about” can mean within 1 or more than 1 standard deviation, per the practice in the art. As another example, “about” can mean a range of up to 20%, up to 10%, up to 5%, or up to 1% of a given value. With respect to biological systems or processes, the term “about” can mean within an order of magnitude, such as within 5-fold or within 2-fold of a value. Where particular values are described in the application and claims, unless otherwise stated, the term “about” means within an acceptable error range for the particular value.

The term “subject,” as used herein, generally refers to a vertebrate, such as a mammal (e.g., a human). Mammals include, but are not limited to, murines, simians, humans, farm animals, sport animals, and pets (e.g., a dog or a cat). Tissues, cells, and their progeny of a biological entity obtained in vivo or cultured in vitro are also encompassed. In some embodiments, the subject is a patient. In some embodiments, the subject is symptomatic with respect to a disease (e.g., cancer). Alternatively, in some cases, the subject is asymptomatic with respect to the disease. In some cases, the subject does not have the disease.

The term “biological sample,” as used herein, generally refers to a sample derived from or obtained from a subject, such as a mammal (e.g., a human). Biological samples are contemplated to include but are not limited to, hair, fingernails, skin, sweat, tears, ocular fluids, nasal swab or nasopharyngeal wash, sputum, throat swab, saliva, mucus, blood, serum, plasma, pleural effusions, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the term “extrachromosomal DNA” or “ecDNA” generally refers to circular DNA that is not found in a chromosome. Extrachromosomal DNA is a distinct entity that is sometimes observed in the nuclei of some cancer cells and, in some cases, carries a driver oncogene. In some cases, extrachromosomal DNA carries more than one copy of a driver oncogene.

As used herein, the term “biomarker” generally refers to a measurable indicator of a biological state or condition. Exemplary biomarkers used in methods and processes herein include, but are not limited to, nucleic acid copy number, co-amplified genes, functional pathway mutations, gene fusions, fragment length, SNP/SNV frequency, indel frequency and location, microsatellite instability, tumor inflammation score, gene expression, such as, PD-L1 expression, and tumor mutational burden.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses that are encompassed within the spirit of the invention as defined by the scope of the claims will occur to those skilled in the art.

### Example 1: Extrachromosomal DNA (ecDNA) is a Biomarker for Insensitivity to Checkpoint Inhibitor Treatment in Gastric Cancer

In the KEYNOTE-059 study, the anti-PD-1 checkpoint inhibitor pembrolizumab was shown to have a modest overall response of 11.6%. Common predictors of response including high microsatellite instability (MSI-H), PD-L1 expression, tumor mutational burden (TMB) or tumor inflammation signature (TIS), were not individually sufficient to enrich for predicted responders. Recent pan-cancer studies have highlighted a unique population of cancer patients whose tumors appear to be driven by oncogene amplifications on extrachromosomal DNA (ecDNA). These ecDNA-driven tumors are aggressive, characterized by high levels of genomic instability, are more aggressive, have a worse prognosis, and are difficult to treat. We sought to understand if tumors that possess ecDNA represent a subset of the patient group that is non-responsive to anti-PD-1 therapy.

In the present example, the ecDNA status was determined for gastric cancer patients (N=108) using whole genome sequencing (WGS) from the TCGA Pan-cancer dataset. These patients had been previously subtyped for EBV status, genomic stability (GS), microsatellite instability (MSI), and chromosomal instability (CIN). Patients that were ecDNA positive were re-classified into a set regardless of gastric subtype. Additionally, TMB, TIS, and PD-L1 expression levels were collected.

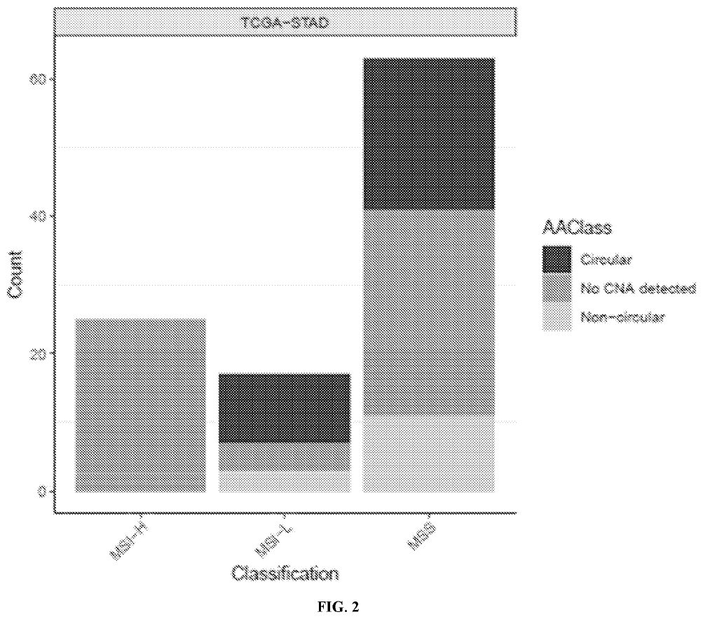

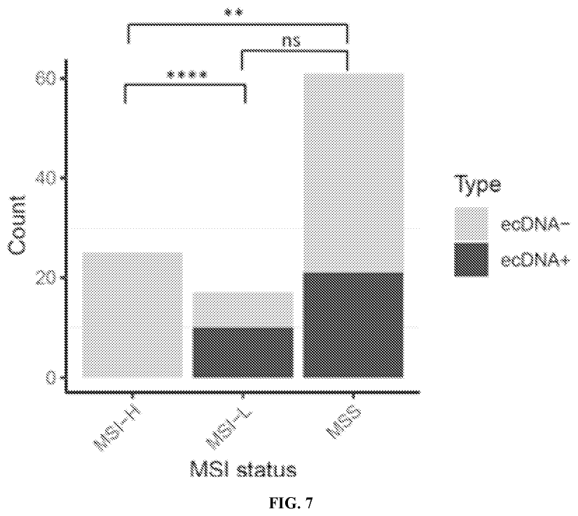

Thirty-two percent of gastric cancer patients were positive for ecDNA signatures and mutually exclusive from the 23% of MSI-H patients (FIG. 2, FIG. 7). MSI status (reported frequently as MSI-H, MSS, or MSI-L) is a frequent biomarker in immune-oncology (IO) therapy (and an approved pan-solid tumor companion diagnostic for pembrolizumab). It was also found that ecDNA positive tumors had statistically significantly lower TIS than all other groups (p-value<0.05) except CIN tumors (p-value=0.09). TIS is proportional to the level of immune activity of a tumor specimen and positively correlates to an improved response to pembrolizumab. The ecDNA positive tumors also had lower PD-L1 expression than all but GS tumors. PD-L1 expression is used as a biomarker for a positive response to pembrolizumab, and specific PD-L1 antibody/scoring combinations are a companion diagnostic for pembrolizumab. Only MSI-H showed statistically significantly higher TMB scores compared to every other group (p-value<0.001); no difference in TMB scores were observed between every other pair of groups.

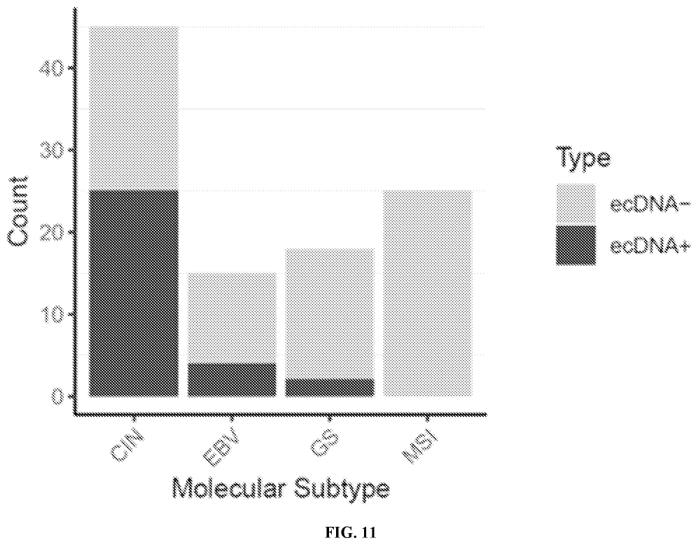

ecDNA+ and ecDNA− status was compared for tumors across various molecular subclasses (FIG. 11). This comparison showed that ecDNA is enriched in pembrolizumab non-responsive CIN patients. Further, ecDNA is reduced or wholly absent in the pembrolizumab responsive subgroup EBV/CIN. This observation illustrates that there could be potential differences in response between the CIN/ecDNA+ subgroup and other subclasses.

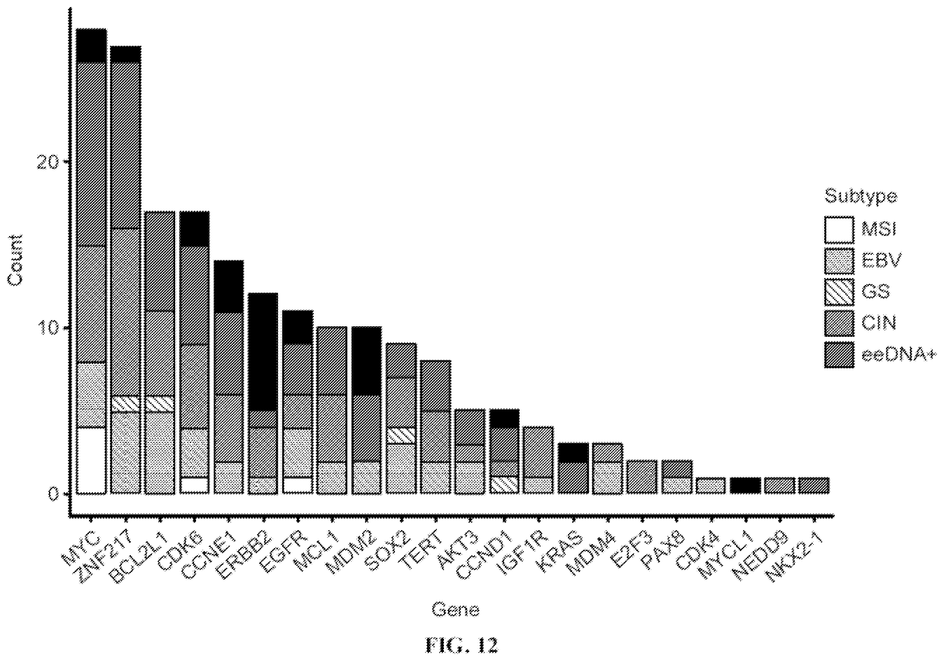

The number of patients were grouped by each subtype that have CNV gain in various oncogene hotspots (FIG. 12). The dark red bar indicates patients where the amplified oncogene has been inferred to be specifically amplified on ecDNA. Notably, several key immune-related oncogenes (MYC, CDK6, CCNE1, CCND1) are frequently found amplified in ecDNA+ patients. Further ERBB2/MDM2 are frequently specifically amplified on ecDNA structure.

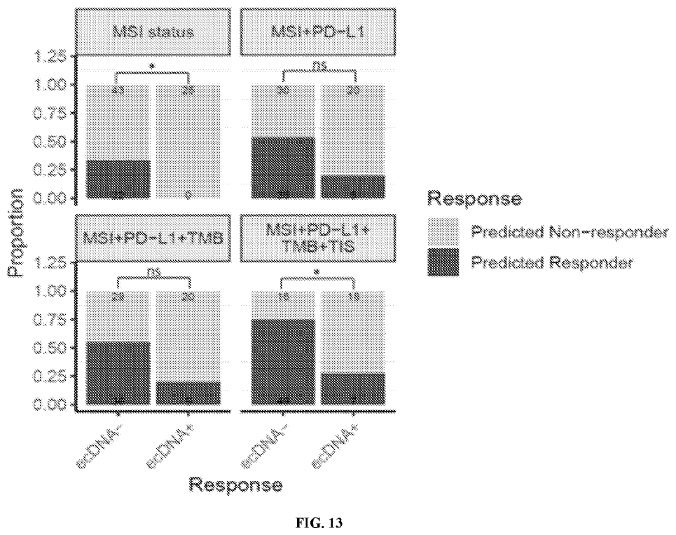

The proportion of ecDNA+ and ecDNA− patients predicted to be responders/non-responders of pembrolizumab was determined using combinations of biomarkers (FIG. 13). In this analysis, ecDNA negative patients were found to be significantly enriched in predicted responders. Likewise, ecDNA positive patients were enriched in predicted non-responders.

These data demonstrate that patients whose tumors are ecDNA positive represent a unique population that displays signatures, including MSS, low TIS, and PD-L1 expression, that are associated with lack of response to checkpoint inhibitor therapy. Thus, the determination of tumor ecDNA status may have utility as an additional patient selection strategy for checkpoint inhibitor therapy. As ecDNA are not limited to gastric cancers, this study highlights the importance of the development of a clinical diagnostic test for ecDNA status and the need for further research on ecDNA biology, its impact on immunotherapy response, and potential ecDNA-directed therapeutics.

### Example 2: Workflow for Analysis of ecDNA Signatures

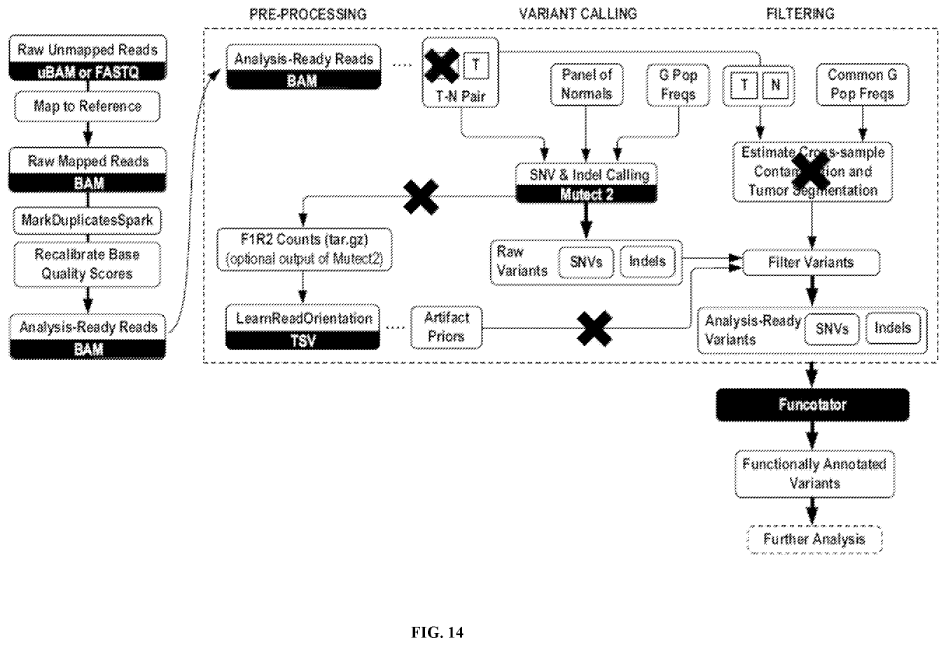

A flow diagram of the method is shown in FIG. 14. Exomes were prepared using Human Core Exome kit (Twist Bioscience) and sequenced on an Illumina platform with 2×150 paired-end read configuration.

Sequencing data was processed according to genome analysis tool kit (GATK) recommended practices (see gatk.groadinstitute.org/hc/en-us/articles/30035894731-Somatic-short-variant-discovery-SVNs-Indels-). Briefly, raw reads were aligned to the human genome (version GRCh37) using Burrows-Wheeler aligner (BWA) minimal exact match (MEM). Small somatic variations were then extracted using Mutect2 (GATK v4.1.9.0) and filtered using FilterMutectCalls.

Downstream analysis of the resulting variant call format (VCF) files was performed in R/Bioconductor.

### Example 3: Analysis of Mutation Rate in ecDNA Regions

Exome data was obtained from a culture of SNU16 cells, and VCF files produced by GATK Mutect2 were used to calculate rates of substitutions, insertions, and deletions in one million nucleotide-long genomic windows over the entire autosomal genome. Mean coverage depth was estimated by averaging depth for all mutations in that particular genomic window as reported in the VCF file.

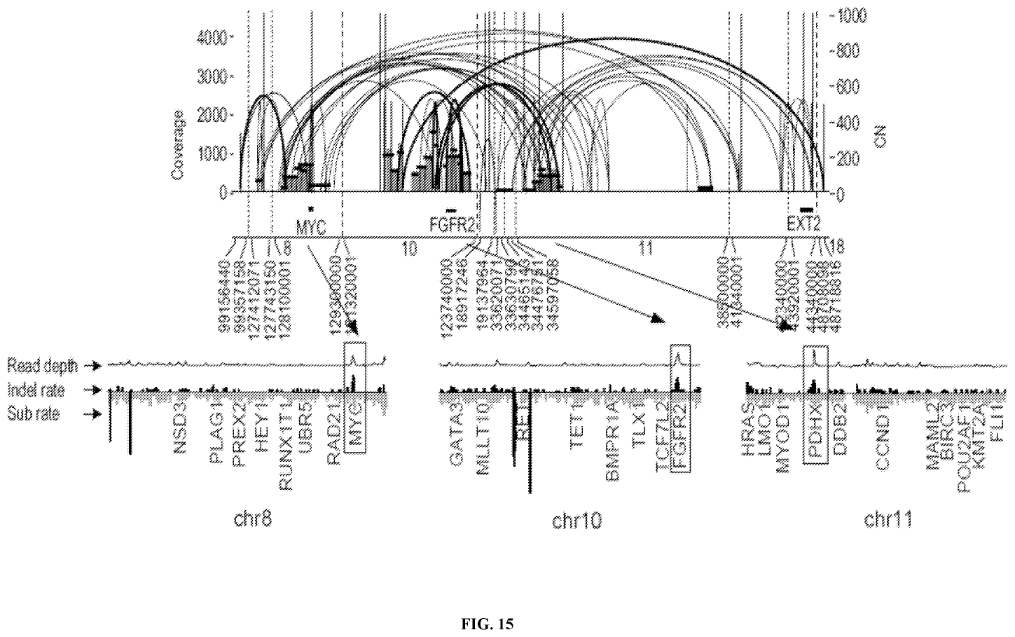

The resulting data showed that ecDNA regions in SNU16 cells are associated with high mutation rates. FIG. 15 shows, in the upper panel, the structure of an ecDNA-containing amplicon present in SNU16 cells as determined using AmpliconArchitect. In the lower panel of FIG. 15, average depth is shown by a line, insertion/deletion (indel) rate is shown by upward bars and substitution rate is shown by downward bars for three genomic regions corresponding to ecDNA as per AmpliconArchitect output, showing increased coverage reflecting copy number amplification of that genomic region, as well as increased mutation rate, especially indels. Mutation rates are calculated for one million nucleotides in 1e5 nucleotide sliding windows.

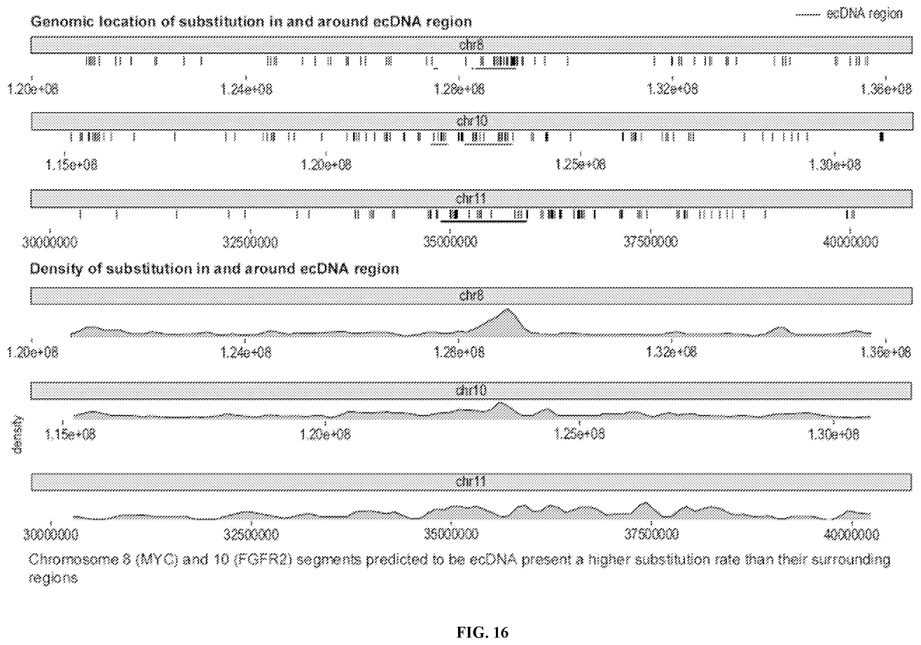

The analysis also showed that substitution rates in ecDNA regions are higher than in surrounding regions. FIG. 16, upper panel, shows location of substitutions in three genomic regions corresponding to ecDNA in SNU16 cells, and their surrounding regions. Each vertical bar represents a unique substitution. Horizontal bars highlight the location of the ecDNA-containing amplicon. In the lower panel of FIG. 16, density of substitutions is shown in three genomic regions corresponding to ecDNA in SNU16 cells, and their surrounding regions (matching genomic coordinates shown in FIG. 16 upper panel). Most ecDNA regions were characterized by a higher rate of substitutions as compared with their surrounding regions.

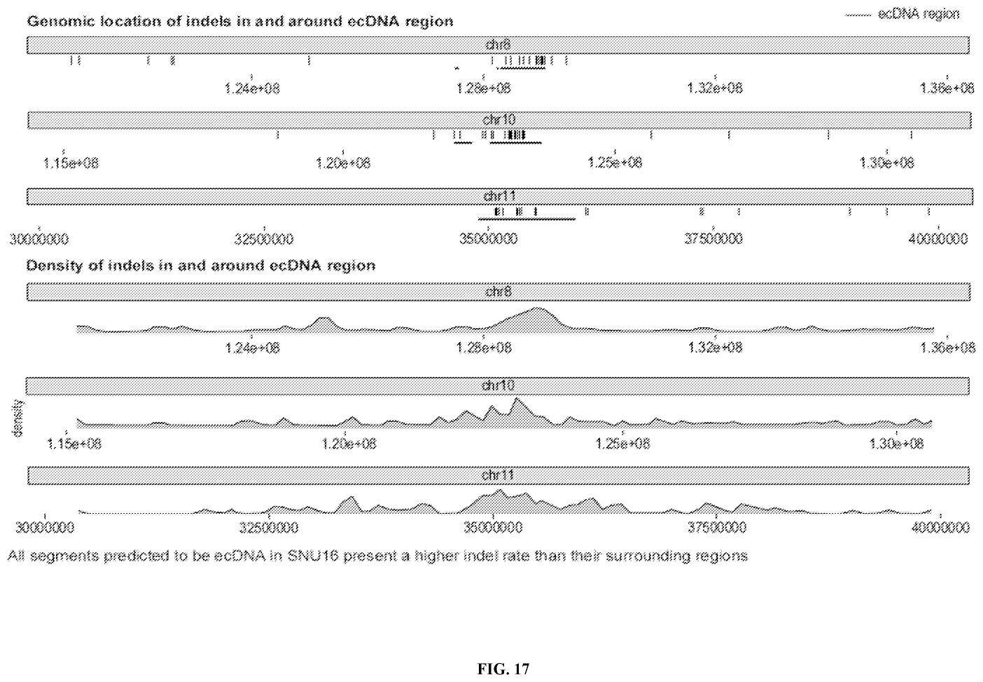

The results of indel rate analysis are shown in FIG. 17. The upper panel of FIG. 17 shows locations of indels in three genomic regions corresponding to ecDNA in SNU61 cells and their surrounding regions. Each vertical bar represents a unique insertion or deletion. The lower panel of FIG. 17 shows the density of indels in three genomic regions corresponding to ecDNA in SNU16 cells and their surrounding regions (matching genomic coordinates show in in FIG. 17 upper panel). These data showed that ecDNA regions were characterized by a higher rate of indels as compared with their surrounding regions.

### Example 4: Comparison of ecDNA and Non-ecDNA Regions

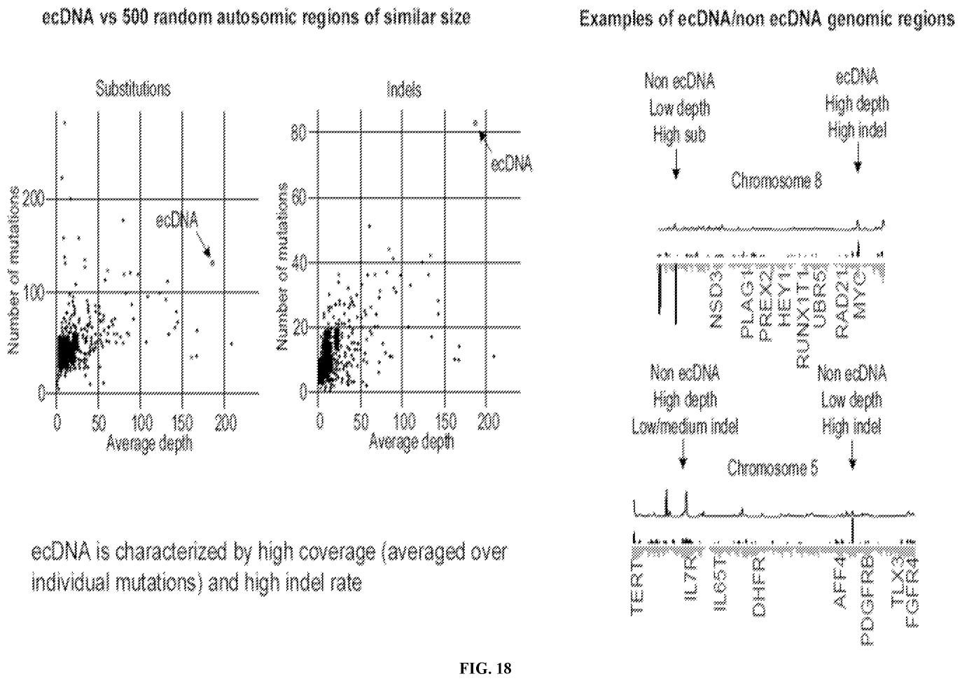

Substitution and indel rates were calculated over the genomic region corresponding to the SNU16 ecDNA amplicon (open dot), as well as over 500 random equally sized continuous genomic regions (closed dots) and plotted against average read depth, showing that ecDNA is characterized by high read depth and indel rate (FIG. 18 left panel). The right panel of FIG. 18 shows average depth (line), indel rate (upward bars), and substitution rate (downward bars) per million nucleotides (1e5 sliding window) exemplifying genomic regions with various combinations of substitution/indel rates and read depth, demonstrating that mutation rate is not necessarily confounding with read depth.

### Example 5: Analysis of Mutations as a Result of Treatment

A culture of SNU16 cells was subjected to a sequence of treatments, starting with infigratinib and followed with erlotinib. Exome data was obtained from these cells at various time points during this experiment: before treatment with infigratinib, two weeks into treatment with 1 μM infigratinib, after 12 weeks of treatment with 1 μM infigratinib, before treatment with erlotinib, two weeks into treatment with 5 μM erlotinib, and four weeks into treatment with 5 μM erlotinib. VCF files produced by GATK Mutect2 were used to calculate rates of substitutions, insertions, and deletions in one million nucleotide-long genomic windows over the entire autosomal genome. Mean coverage depth was estimated by averaging depth for all mutations in that particular genomic window as reported in the VCF file.

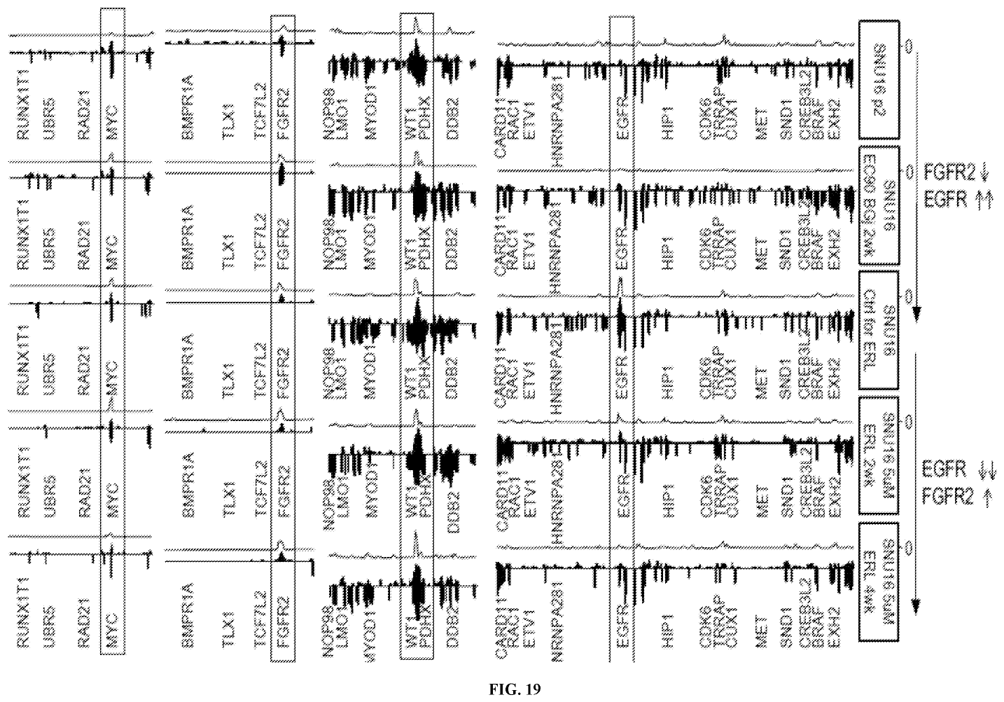

Data in FIG. 19 shows MYC and PDHX ecDNA copy numbers were mostly stable across treatment. FGFR2 ecDNA copy number moderately decreased during treatment with infigratinib. EGFR ecDNA copy number dramatically increased during treatment with infigratinib but went back down upon treatment with erlotinib. Boxes highlight the genomic locations corresponding to these ecDNA and show that indel rates followed the same trends as ecDNA copy numbers.

### Example 6: Analysis of Mutations in H2170 Cells

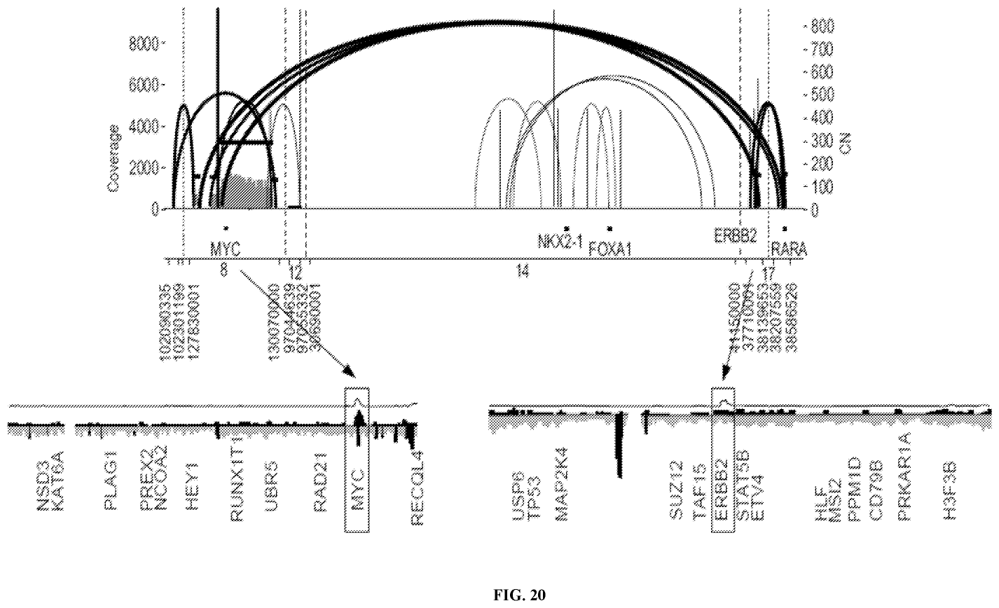

The analysis of mutations seen in H2170 cells is shown in FIG. 20. The upper panel shows the structure of an ecDNA-containing amplicon present in H2170 squamous cell carcinoma cells (ATCC) as determined using AmpliconArchitect. In the lower panel of FIG. 20, average depth (line) indel rate (upward bars), and substitution rate (downward bars) are shown per million nucleotides (1e5 sliding window) in genomic regions corresponding to ecDNA as per AmpliconArchitect output. This data showed increased read depth reflecting copy number amplification and slightly (ERBB2) to dramatically (MYC) increased indel rate.

### Example 7: Functional Pathway Analysis

The P53 pathway is a tumor suppressive pathway that can signal cellular apoptosis in response to oncogene activation or DNA damage. Mutations in tumor suppressor gene TP53 or amplification in MDM2, which represses TP53, can disable the pathway. Table 1 shows that ecDNA+ patients are statistically significantly likely to have P53 pathway alterations compared to ecDNA− patients.

TABLE 1
Alterations in the P53 pathway are enriched in predicted ecDNA+
patients compared to predicted ecDNA− patients

| | TP53mut or MDM2-amp | TP53wt and MDM2-noamp |
|---|---|---|
| ecDNA+ | 205 | 101 |
| ecDNA− | 469 | 832 |

Data was obtained from the pancancer TCGA database based upon 1607 cancer patients for which ecDNA status (Kim, H., et al. (2020). “Extrachromosomal DNA is associated with oncogene amplification and poor outcome across multiple cancers.” Nature Genetics.), TP53 mutation status (Gao, J., et al. (2013). “Integrative analysis of complex cancer genomics and clinical profiles using the cBioPortal.” Sci Signal 6(269): pl 1.), and MDM2 amplification status (www.cancer.gov/tcga) was known. ecDNA status was determined by running AmpliconArchitect on WGS data. TP53 mutation status was inferred from WGS and WXS data. MDM2 amplification status was determined from Affymetrix SNP 6.0 array. For purposes of this analysis, copy number of 3 or greater was considered amplified. P53 pathway altered patients herein were classified as any patient with TP53 mutation or MDM2 amplification. All other patients were considered P53 pathway competent. 67% of patients inferred as ecDNA+ in this analysis had P53 pathway alterations compared to 36% of the inferred ecDNA− patients (Pearson's Chi-squared; p-value<1e-15).

Microsatellite instability (MSI) is a condition in which genes that regulate DNA mismatch repair do not function correctly, resulting in a high number of mutations in the genome. As shown below, ecDNA+ cancers inversely correlate with MSI status (Table 2).

TABLE 2
MSI status of predicted ecDNA+ patients compared to predicted ecDNA− patients

| | MSI-H | MSS or MSI-L |
|---|---|---|
| ecDNA+ | 4 | 326 |
| ecDNA− | 91 | 1400 |

Data was obtained from the pancancer TCGA database and based upon 1821 cancer patients for which ecDNA status (Kim, H., et al. (2020). “Extrachromosomal DNA is associated with oncogene amplification and poor outcome across multiple cancers.” Nature Genetics.), TP53 mutation status (Gao, J., et al. (2013). “Integrative analysis of complex cancer genomics and clinical profiles using the cBioPortal.” Sci Signal 6(269): pl 1.) and MSI status (Kautto, E. A., et al. (2017). “Performance evaluation for rapid detection of pan-cancer microsatellite instability with MANTIS.” Oncotarget 8(5): 7452-7463) was known. ecDNA status was inferred by running AmpliconArchitect on WGS data. MSI status was inferred by running MANTIS4 on WXS data (Bonneville, R., et al. (2017). “Landscape of Microsatellite Instability Across 39 Cancer Types.” JCO Precis Oncol 2017). 96% of MSI-H patients were ecDNA-, and ecDNA− patients were seven times more likely to be MSI-H compared to ecDNA+ patients. This result shows that ecDNA positivity is anti-correlated to MSI status, and is statistically significant (Pearson's Chi-squared; p-value<0.0006).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of treating a cancer, the method comprising (a) obtaining biomarker data for a biological sample derived from a subject diagnosed or suspected of having a cancer; (b) classifying the ecDNA status of the sample as ecDNA positive or ecDNA negative; and (c) determining a treatment plan on the basis of the ecDNA status.

2. The method of claim 1, wherein the treatment plan excludes the administration of an immune checkpoint inhibitor when the sample is ecDNA positive.

3. The method of claim 1 or claim 2, wherein the treatment plan excludes the administration of an immune checkpoint inhibitor as a first line therapy when the sample is ecDNA positive.

4. The method of claim 1, wherein the treatment plan includes the administration of an immune checkpoint inhibitor when the sample is ecDNA negative.

5. The method of any one of claims 1 to 4, wherein the biomarker data is selected from the group consisting of nucleic acid copy number, co-amplified genes, gene fusions, fragment length, SNP/SNV frequency, indel frequency, indel location, a functional pathway mutation, microsatellite instability, tumor inflammation score, PD-L1 expression, or tumor mutational burden.

6. A method of classifying potential for responsiveness of a subject to immune checkpoint therapy, comprising determining an ecDNA positive status of a sample taken from the subject, wherein the ecDNA positive status is determined based on a threshold of an ecDNA signature; and classifying the subject as potentially responsive to immune checkpoint therapy when the sample is below threshold for the ecDNA positive status.

7. A method of classifying potential for non-responsiveness of a subject to immune checkpoint therapy, comprising determining the ecDNA positive status of a sample taken from the subject, wherein the ecDNA positive status is determined based on a threshold of an ecDNA signature; and classifying the subject as potentially nonresponsive to immune checkpoint therapy when the sample is above the threshold for the ecDNA positive status.

8. The method of claim 6 or claim 7, wherein the ecDNA signature is selected from the group consisting of nucleic acid copy number, co-amplified genes, gene fusions, fragment length, SNP/SNV frequency, indel frequency, indel location, a functional pathway mutation, microsatellite instability, tumor inflammation score, PD-L1 expression, or tumor mutational burden.

9. The method of any one of claims 1 to 8, wherein the sample comprises tumor cells, circulating tumor cells, circulating vesicles, or circulating tumor DNA.

10. The method of any one of claims 1 to 9, wherein the sample is blood, serum, plasma, lymph, pleural effusion, saliva, urine, stool, tissue, resected tumor, or a combination thereof.

11. A method of detecting an ecDNA positive cancer, the method comprising: (a) determining a first frequency of base substitution or indels within a defined region of DNA from the sample; (b) generating a comparison of the first frequency to a control frequency; and (c) classifying an ecDNA status of the sample based on the comparison.

12. The method of claim 11, wherein an increase in the first frequency as compared to the control frequency indicates an ecDNA positive sample.

13. The method of claim 11, wherein (c) further comprises assessing presence of one or more ecDNA signatures and classifying the ecDNA status based on the one or more ecDNA signatures and the comparison of frequencies.

14. The method of claim 13, wherein the one or more ecDNA signatures are selected from the group consisting of nucleic acid copy number, co-amplified genes, gene fusions, fragment length, SNP/SNV frequency, indel frequency, indel location, a functional pathway mutation, microsatellite instability, tumor inflammation score, PD-L1 expression, or tumor mutational burden.

15. A method of assessing progression of a cancer treatment, the method comprising: (a) determining a first frequency of base substitutions or indels within a defined region of DNA from a first sample from a subject; (b) determining a second frequency of base substitutions or indels within the defined region of DNA from a second sample from the subject, wherein the second sample is obtained after treatment with a targeted cancer therapy agent; and (c) comparing the first frequency to a second frequency to identify a change in the ecDNA status of the sample.

16. The method of claim 15, wherein the change in ecDNA status comprises an increase in the second frequency as compared to the first frequency.

17. The method of claim 16, further comprising changing the cancer treatment based on the change in ecDNA status.

18. The method of any one of claims 11 to 17, wherein the sample comprises tumor cells, circulating tumor cells, circulating vesicles, or circulating tumor DNA.

19. The method of any one of claims 11 to 18, wherein the sample is blood, serum, plasma, lymph, pleural effusion, saliva, urine, stool, tissue, resected tumor, or a combination thereof.

20. A method of assessing the potential for resistance to a cancer treatment, the method comprising: (a) determining a first frequency and location of base substitution or indels within a defined region of DNA from a first sample from a subject; (b) determining a second frequency and location of base substitution or indels within the defined region of DNA from a second sample from the subject, wherein the second sample is obtained during a course of treatment with a targeted cancer therapy agent; (c) comparing the first frequency and location to a second frequency and location, wherein the subject is determined to have the potential for resistance to a cancer treatment when the second frequency or location is changed as compared to the first frequency or location.

21. The method of claim 20, wherein the first sample, the second sample or both the first sample and second sample comprise tumor cells, circulating tumor cells, circulating vesicles, or circulating tumor DNA.

22. The method of claim 20 or claim 21, wherein the first sample, the second sample, or both the first sample and the second sample are blood, serum, plasma, lymph, pleural effusion, saliva, urine, stool, tissue, resected tumor, or a combination thereof.

23. A method of detecting an ecDNA positive cancer in a subject, the method comprising: (a) obtaining biomarker data for a biological sample derived from the subject; (b) processing the biomarker data from the subject with control biomarker data; and (c) classifying the subject as having an ecDNA positive cancer when, based on the result of (b), the biomarker data from the subject is significantly different as compared to the control biomarker data.

24. The method of claim 22, wherein the control biomarker data is derived from subjects having ecDNA negative cancer or from a non-cancerous source.

25. The method of claim 22, wherein the control biomarker data is a predetermined threshold value.

26. The method of any one of claims 22 to 25, wherein the biomarker data comprises one or more of nucleic acid copy number, co-amplified genes, gene fusions, fragment length, SNP/SNV frequency, indel frequency, indel location, microsatellite instability, tumor inflammation score, PD-L1 expression, a functional pathway mutation, or tumor mutational burden.

27. The method of any one of claims 22 to 26, wherein the biomarker data is measured by whole exome sequencing, whole genome sequencing (WGS), or targeted sequencing.

28. The method of claim 26, wherein the tumor inflammation score is measured in a gene expression assay on a cancer cell from the subject.

29. The method of claim 26, wherein the PD-L1 expression level is measured by a PD-L1 RNA gene expression assay on a cancer cell from the subject.

30. The method of claim 26, wherein the PD-L1 expression level is measured by a PD-L1 protein expression assay on a cancer cell from the subject.

31. The method of claim 26, wherein the microsatellite instability is measured by sequencing nucleic acids derived from a cancer cell, a tumor, a circulating tumor cell, a circulating vesicle, or circulating tumor DNA from the subject.

32. The method of claim 26, wherein the tumor mutational burden is measured by sequencing nucleic acids derived from a cancer cell from the subject.

33. The method of any one of claims 1 to 32, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer.

34. The method of any one of claims 1 to 33, wherein the method further comprises administering a therapeutic agent to the subject.

35. The method of claim 34, wherein the therapeutic agent comprises atezolizumab, avelumab, cemiplimab, durvalumab, nivolumab, or pembrolizumab.

36. The method of claim 34, wherein the therapeutic agent does not comprise an anti-PD-L1 or anti-PD-1 antibody.

37. A method of detecting an ecDNA positive cancer in a subject, the method comprising: (a) obtaining a tumor inflammation score for a biological sample derived from the subject; (b) computer processing the tumor inflammation score from the subject with a control tumor inflammation score; and (c) classifying the subject as having an ecDNA positive cancer when, based on the result of (b), the tumor inflammation score from the subject is decreased as compared with the control tumor inflammation score.

38. The method of claim 37, wherein the method further comprises: (d) obtaining a PD-L1 expression level for the biological sample derived from the subject; (e) computer processing the PD-L1 expression level from the subject with a control PD-L1 expression level; and (f) classifying the subject as having an ecDNA positive cancer when based on the results of (b) and (e), the tumor inflammation score from the subject is decreased as compared with the control tumor inflammation and the PD-L1 expression level is decreased as compared to the control PD-L1 expression level.

39. The method of claim 37 or claim 38, wherein the control tumor inflammation score or the control PD-L1 expression level is derived from a subject that has ecDNA negative cancer or from a non-cancerous source.

40. The method of any one of claims 37 to 39, wherein the tumor inflammation score is measured in a gene expression assay on a cancer cell from the subject.

41. The method of any one of claims 36 to 40, wherein the PD-L1 expression level is measured by measuring a PD-L1 RNA level in a cancer cell from the subject.

42. The method of any one of claims 36 to 40, wherein the PD-L1 expression level is measured by measuring a PD-L1 protein level in a cancer cell from the subject.

43. The method of any one of claims 37 to 42, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer.

44. The method of any one of claims 37 to 43, wherein the cancer is gastric cancer.

45. The method of any one of claims 37 to 44, wherein the method further comprises administering a therapeutic agent to the subject.

46. The method of claim 45, wherein the therapeutic agent comprises atezolizumab, avelumab, cemiplimab, durvalumab, nivolumab, or pembrolizumab.

47. The method of claim 45, wherein the therapeutic agent does not comprise an anti-PD-L1 or anti-PD-1 antibody.

48. A method of detecting an ecDNA positive cancer in a subject, the method comprising: (a) obtaining a PD-L1 expression level for the biological sample derived from the subject; (b) computer processing the PD-L1 expression level from the subject with a control PD-L1 expression level; and (c) classifying the subject as having an ecDNA positive cancer when based on the results of (b), the PD-L1 expression level is decreased as compared to the control PD-L1 expression level.

49. The method of claim 48, wherein the method further comprises: (d) obtaining a tumor inflammation score for a biological sample derived from the subject; (e) computer processing the tumor inflammation score from the subject with a control tumor inflammation score; and (0 classifying the subject as having an ecDNA positive cancer when based on the result of (b) and (e), the PD-L1 expression level is decreased as compared to the control PD-L1 expression level and the tumor inflammation score from the subject is decreased as compared with the control tumor inflammation score.

50. The method of claim 48 or claim 49, wherein the control tumor inflammation score or the control PD-L1 expression level is derived from a subject that has ecDNA negative cancer or from a non-cancerous source.

51. The method of any one of claims 48 to 50, wherein the PD-L1 expression level is measured by measuring a PD-L1 RNA level in a cancer cell from the subject.

52. The method of any one of claims 48 to 50, wherein the PD-L1 expression level is measured by measuring a PD-L1 protein level in a cancer cell from the subject.

53. The method of any one of claims 47 to 52, wherein the tumor inflammation score is measured in a gene expression assay on a cancer cell from the subject.

54. The method of any one of claims 48 to 53, wherein the cancer is selected from the group consisting of colon cancer, non-small cell lung cancer, small cell lung cancer, breast cancer, hepatocellular carcinoma, liver cancer, skin cancer, malignant melanoma, endometrial cancer, esophageal cancer, soft tissue sarcoma, gastric cancer, ovarian cancer, pancreatic cancer, and brain cancer.

55. The method of any one of claims 48 to 54, wherein the cancer is gastric cancer.

56. The method of any one of claims 48 to 55, wherein the method further comprises administering a therapeutic agent to the subject.

57. The method of claim 56, wherein the therapeutic agent comprises atezolizumab, avelumab, cemiplimab, durvalumab, nivolumab, or pembrolizumab.

58. The method of claim 56, wherein the therapeutic agent does not comprise an anti-PD-L1 or anti-PD-1 antibody.
